(19) United States
(12) Patent Application Publication
(10) Pub. No.: US 2024/0076391 A1
(43) Pub. Date: Mar. 7, 2024

(54) HUMAN CCR8 BINDERS

(71) Applicants: ONCURIOUS NV, Leuven (BE); VIB VZW, Gent (BE); VRIJE UNIVERSITEIT BRUSSEL, Brussel (BE)

(72) Inventors: Hui Qi Lu, Gent (BE); Catelijne Stortelers, Gent (BE); Nadia Van Boxel, Gent (BE); Shanna Van Zwam, Gent (BE); Jimmy Borloo, Gent (BE); Bruno Dombrecht, Gent (BE); Jo Van Ginderachter, Gent (BE); Pascal Merchiers, Leuven (BE); Rosa Martin, Gent (BE)

(21) Appl. No.: 18/259,155

(22) PCT Filed: Dec. 23, 2021
(86) PCT No.: PCT/EP2021/087506

§ 371 Date: Jun. 23, 2023

(30) Foreign Application Priority Data
Dec. 24, 2020 (EP) 20217315.9
Apr. 7, 2021 (EP) 21167288.6

(51) Int. Cl. C07K 16/28; A61K 45/06

(52) U.S. Cl. CPC C07K 16/2866; A61K 45/06; A61K 2039/505; C07K 2317/33; C07K 2317/569; C07K 2317/72; C07K 2317/732; C07K 2317/734

(57) ABSTRACT

The present invention relates to human CCR8 (hCCR8) binders, wherein the hCCR8 binder is cross-reactive with a non-human primate CCR8. Such binders are particularly useful for the depletion of intra-tumoural regulatory T-cells and immunotherapy in general.

### CROSS-REFERENCE TO RELATED APPLICATIONS

This application is a national phase entry under 35 U.S.C. § 371 of International Patent Application PCT/EP2021/087506, filed Dec. 23, 2021, designating the United States of America and published in English as International Patent Publication WO 2022/136647 on Jun. 30, 2022, which claims the benefit under Article 8 of the Patent Cooperation Treaty to European Patent Application Serial No. 20217315.9, filed Dec. 24, 2020, and European Patent Application Serial No. 21167288.6, filed Apr. 7, 2021, the entireties of which are hereby incorporated by reference.

### FIELD OF THE INVENTION

The present invention relates to human CCR8 (hCCR8) binders. Such binders are particularly useful for the depletion of intra-tumoural regulatory T-cells and immunotherapy in general.

### BACKGROUND OF THE INVENTION

Regulatory T (Treg) cells are one of the integral components of the adaptive immune system whereby they contribute to maintaining tolerance to self-antigens and preventing auto-immune diseases. However, Treg cells are also found to be highly enriched in the tumour microenvironment of many different cancers (Colombo and Piconese, 2007; Nishikawa and Sakaguchi, 2014; Roychoudhuri et al., 2015). In the tumour microenvironment, Treg cells contribute to immune escape by reducing tumour-associated antigen (TAA)-specific T-cell immunity, thereby preventing effective anti-tumour activity. High tumour infiltration by Tregs is hence often associated with an invasive phenotype and poor prognosis in cancer patients (Shang et al., 2015; Plitas et al., 2016).

Acknowledging the significance of tumour-infiltrating Treg cells and their potential role in inhibiting anti-tumour immunity, multiple strategies have been proposed to modulate Treg cells in the tumour microenvironment. Several studies have demonstrated that modulating Tregs has the potential to offer significant therapeutic benefit (Elpek et al, 2007).

However, one major challenge associated with Treg modulation is that systemic removal or inhibition of Treg cells may elicit autoimmunity. It is therefore critical to specifically deplete tumour-infiltrating Treg cells while preserving tumour-reactive effector T cells and peripheral Treg cells (e.g. circulating blood Treg cells) in order to prevent autoimmunity.

Wang et al. (PloSONE 2012, e30793) reported increased expression of CCR8 on tumour-infiltrating FoxP3+ T-cells and suggested that blocking CCR8 may lead to the inhibition of migration of Tregs into the tumours. Due to the high and relatively specific expression of CCR8 on tumour-infiltrating Tregs, neutralizing monoclonal antibodies against CCR8 have been suggested for the modulation and depletion of this Treg population in the treatment of cancer (EP3431105 A1 and WO2019/157098 A1). WO2018/181425 suggests that, in mice, a neutralizing anti-CCR8 mAb is able to deplete Treg cells in tumour tissues by antibody-dependent cell-mediated cytotoxicity (ADCC), and thereby enhance tumour immunity. Through their neutralizing activity, these antibodies inhibit Treg migration into the tumour, reverse the suppressive function of Tregs and deplete intratumoural Tregs (WO2019/157098 A1). Recently, Wang et al. (Cancer Immunol Immonother 2020, https://doi.org/10.1007/s00262-020-02583-y) showed that CCR8 blockade could destabilize intratumoural Tregs into a fragile phenotype accompanied with reactivation of the antitumour immunity and augment anti-PD-1 therapeutic benefits.

Neutralizing monoclonal antibodies against human CCR8 have been described in WO2007044756 A2, including the preferred antibody 433H. EP3616720 A1 shows ADCC dependent Treg depletion using an antibody against murine CCR8 in a mouse model enhances tumor immunity. However, as the antibody does not bind human CCR8, it cannot be developed for human cancer therapy. Opposite thereto, WO2020138489 A1 from the same applicant provides neutralizing antibodies with ADCC activity against human CCR8 for cancer treatment. Its humanized antibodies bind to human CCR8 and neutralize CCL1-induced calcium influx. It is indicated that binding to the N-terminal region of human CCR8 is in important element for exerting neutralizing activity. Interestingly, the blocking anti-CCR8 antibodies of the prior art, such as WO2007044756 A2 and EP3616720 A1, indeed all bind the N-terminal region of CCR8.

There is a need for new binders to human CCR8, allowing for the development of new cancer treatments.

### SUMMARY OF THE INVENTION

The inventors have now identified novel binders of human CCR8 (hCCR8). Interestingly, despite the teaching of the prior art that N-terminal binding to CCR8 is required for the neutralizing activity of anti-CCR8 antibodies, the binders of the present invention do show blocking activity, but bind to extracellular loops of CCR8. Additionally, it has been found that these binders are cross-reactive with non-human primate CCR8 thereby allowing for in vivo testing in animal models. Thus, the inventors have found that cross-reactive neutralizing binders can be generated by binding the extracellular loops of CCR8. In particular, it has been found that extracellular loops 2 and 3 of CCR8 are a suitable target for the generation of human CCR8 binders. It is thus an object of the invention to provide blocking hCCR8 binders which are cross-reactive with non-human primate CCR8. Therefore in a first embodiment, the present invention provides an hCCR8 binder, wherein said hCCR8 binder is cross-reactive with non-human primate CCR8, preferably a cynomologus macaque (Macaca fascicularis) CCR8 or a rhesus macaque (Macaca mulatta) CCR8.

Preferably, the hCCR8 binder binds to an extracellular loop of hCCR8. Preferably, the hCCR8 binds to extracellular loop 2 or to extracellular loop 3 of hCCR8.

In a further embodiment, the hCCR8 binder comprises a single-domain antibody moiety that binds to hCCR8.

In still a further embodiment, the single-domain antibody moiety that binds to hCCR8 comprising three complementary determining regions (CDRs), namely CDR1, CDR2 and CDR3, wherein CDR3 is selected from the group consisting of (a) the amino acid sequence of NARARLWSVFDY (SEQ ID NO: 10), (b) the amino acid sequence of NAKTVTRTGGVITGSRIEYDY (SEQ ID NO: 11), (c) the amino acid sequence of YSKSLIGTSRYEI (SEQ ID NO: 12), (d) the amino acid sequence of NVKTIKRTGGILTGSKIEYDY (SEQ ID NO: 13), (e) amino acid sequences having at least 80% amino acid sequence identity with SEQ ID NO: 10, 11, 12 or 13, and (f) amino acid sequences having 3, 2 or 1 amino acid different from SEQ ID NO: 10, 11, 12 or 13.

Preferably, CDR1 is selected from the group consisting of (a) the amino acid sequence of GFSFSSFA (SEQ ID NO: 1), (b) the amino acid sequence of GRAFSSYN (SEQ ID NO: 2), (c) the amino acid sequence of RSIYNVLA (SEQ ID NO: 3), (d) the amino acid sequence of RSIYNVRA (SEQ ID NO: 4), (e) the amino acid sequence of GSTFSIKS (SEQ ID NO: 5), (f) amino acid sequences having at least 80% amino acid identity with SEQ ID NO: 1, 2, 3, 4 or 5, and (g) amino acid sequences having 3, 2, 1 amino acid difference from SEQ ID NO: 1, 2, 3, 4 or 5, and CDR2 is selected from the group consisting of (a) the amino acid sequence of the amino acid sequence of ITTTGAT (SEQ ID NO: 6), (b) the amino acid sequence of ISSSGT (SEQ ID NO: 7), (c) the amino acid sequence of VWSSGNT (SEQ ID NO: 8), (d) the amino acid sequence of ITRGAGI (SEQ ID NO: 9), (e) amino acid sequences having at least 80% amino acid identity with SEQ ID NO: 6, 7, 8 or 9, and (f) amino acid sequences having 3, 2, 1 amino acid difference from SEQ ID NO: 6, 7, 8 or 9.

In still another embodiment, the single-domain antibody moiety further comprises four framework regions (FRs) having at least 50%, preferably at least 60%, more preferably at least 70%, still more preferably at least 80%, more preferably at least 85% sequence identity to SEQ ID NO: 14 to 20.

In yet another embodiment, the single-domain antibody moiety comprises the amino acid sequence of SEQ ID NO: 21, or SEQ ID NO: 22, or SEQ ID NO: 23, or SEQ ID NO: 24, or SEQ ID NO: 25.

In still another embodiment, the hCCR8 binder comprises a single-domain antibody moiety that binds to human CCR8 and further comprises at least one cytotoxic moiety.

Preferably, the cytotoxic moiety induces antibody-dependent cellular cytotoxicity (ADCC), induces antibody-dependent cellular phagocytosis (ADCP), induces complement-dependent cytotoxicity (CDC), binds to and activates T-cells, or comprises a cytotoxic payload.

Preferably, the cytotoxic moiety comprises a fragment crystallisable (Fc) region moiety.

More preferably, the Fc region moiety has been engineered to increase ADCC, CDC, and/or ADCP activity, such as through afucosylation or by comprising an ADCC, CDC and/or ADCP-increasing mutation.

Another object of the present invention is to provide nucleic acids encoding the hCCR8 binder.

Yet another object of the present invention is to provide hCCR8 binders for use as a medicine.

A further object of the present invention is to provide hCCR8 binders for use in the treatment of a tumour. Preferably, the tumour is selected from the group consisting of breast cancer, uterine corpus cancer, lung cancer, stomach cancer, head and neck cancer, squamous cell carcinoma, skin cancer, colorectal cancer, kidney cancer and T cell lymphoma.

Preferably, the administration of the hCCR8 binder leads to the depletion of tumour-infiltrating regulatory T-cells (Tregs).

In yet a further embodiment, the treatment further comprises administration of a checkpoint inhibitor. A checkpoint inhibitor is a compound that blocks checkpoint proteins from binding to their partner proteins thereby activating the immune system function. Preferably the checkpoint inhibitor blocks proteins selected from the group consisting of PD-1, PD-L1, CTLA-4, TIGIT, TIM-3, LAG-3, VISTA, B7-1, and B7-2. More preferably the checkpoint inhibitor blocks PD-1 or PD-L1.

### BRIEF DESCRIPTION OF FIGURES

FIG. 1 illustrates the evaluation by flow cytometry of four VHHs (VHH-56, VHH-62, VHH-65 and VHH-74) derived from llama immunization with human CCR8 for their binding to human CCR8 stably expressed in HEK293 cells.

FIG. 2 illustrates the evaluation by flow cytometry of four VHHs (VHH-56, VHH-62, VHH-65 and VHH-74) for their binding to Macaca CCR8 stably expressed in HEK293 cells.

FIG. 3 shows the evaluation by flow cytometry of four VHHs (VHH-56, VHH-62, VHH-65 and VHH-74) for their binding to Macaca CCR8 transiently expressed in HEK293 cells.

FIG. 4 shows evaluation of VHH-56, VHH-62, VHH-65 and VHH-74 for their potential to functionally inhibit the action of the human CCL1 ligand on cAMP accumulation in CHO-K1 cells stably expressing recombinant CCR8.

FIG. 5 shows the evaluation of the twelve VHH-Fc fusions VHH-Fc-200, VHH-Fc-201, VHH-Fc-202, VHH-Fc-206, VHH-Fc-207, VHH-Fc-208, VHH-Fc-212, VHH-Fc-213, VHH-Fc-214, VHH-Fc-221, VHFc-222 and VHH-Fc-223 for their binding to human CCR8 on stably transfected in HEK293 cells, in comparison with two control anti-CCR8 mAbs.

FIG. 6 shows the evaluation of VHH-Fc fusions VHH-Fc-200 (SEQ ID NO: 53), VHH-Fc-201 (SEQ ID NO: 62), VHH-Fc-202 (SEQ ID NO: 71), VHH-Fc-206 (SEQ ID NO: 51), VHH-Fc-207 (SEQ ID NO: 60), VHH-Fc-208 (SEQ ID NO: 69), VHH-Fc-212 (SEQ ID NO: 52), VHH-Fc-213 (SEQ ID NO: 61), VHH-Fc-214 (SEQ ID NO: 70), VHH-Fc-221 (SEQ ID NO: 54), VHH-Fc-222 (SEQ ID NO: 63) and VHH-Fc-223 (SEQ ID NO: 72) for their binding to Macaca CCR8 transiently overexpressed in HEK293 cells, in comparison with two control anti-CCR8 mAbs.

FIG. 7 displays evaluation of four VHH-Fc fusions VHH-Fc-201, VHH-Fc-207 VHH-Fc-213 and VHH-Fc-222 for their binding to Macaca CCR8 stably expressed in HEK293T cells, in comparison with two control anti-CCR8 mAbs.

FIG. 8 presents the amino acid sequence of VHH-62 (SEQ ID NO: 35), VHH-65 (SEQ ID NO: 36), VHH-56 (SEQ ID NO37) and VHH-74 (SEQ ID NO: 38). Complementarity determining regions (CDRs) identified using the IMGT method are underlined, whereas CDRs identified using the Kabat method are represented in bold. Asterisks indicate amino acids which are mutated in the optimized cross-reactive hCCR8 binders VHH-81 (SEQ ID NO: 21), VHH-89 (SEQ ID NO: 22), VHH-114 (SEQ ID NO: 23), VHH-115 (SEQ ID NO: 24), and VHH-125 (SEQ ID NO: 25).

FIG. 9 shows the evaluation of the PBMC mediated ADCC activity of both an afucosylated (AF) and a non-afucosylated version of VHH-Fc-268 (SEQ ID NO: 59) and VHH-Fc-269 (SEQ ID NO: 68) in comparison to isotype on hCCR8-expressing HEK292 cells.

### DETAILED DESCRIPTION OF THE INVENTION

The present invention will be described in the following with respect to particular embodiments and with reference to certain drawings but the invention is not limited thereto.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclature used in connection with, and techniques of, molecular biology, immunology, microbiology, genetics and protein and nucleic acid chemistry described herein are those well-known and commonly used in the art.

As described herein before, the present invention provides certain human CCR8 (hCCR8) binders, such as hCCR8 binders, particularly blocking CCR8 binders, that bind to one or more extracellular loops of hCCR8, in particular binders that bind to extracellular loop 2 or extracellular loop 3 of CCR8. Such compounds are particularly useful due to their ability to bind to human CCR8 expressed on a cell, such as a regulatory T-cell, particularly an intra-tumoural regulatory T-cell, and to deplete such cells through their cytotoxic activity. CCR8 is a member of the beta-chemokine receptor family which is predicted to be a seven transmembrane protein similar to G-coupled receptors. Identified ligands of CCR8 include its natural cognate ligand CCL1 (1-309). Human CCR8 received UniProt Knowledgebase entry number P51685. On the other hand, CCR8 from the non-human primate Rhesus macaque (Macaca mulatta) received UniProt Knowledgebase entry number 097665, whereas CCR8 from the non-human primate Cynomolgus macaque (Macaca fascicularis) received UniProt Knowledgebase entry number G7NYJ2.

CCR8 Binders

As described herein, the term “binder” of a specific antigen denotes a molecule capable of specific binding to said antigen. Specifically, a human CCR8 binder as used herein refers to a molecule capable of specifically binding to hCCR8. Such a binder is also referred to herein as a “hCCR8 binder”.

A “cross-reactive binder” as used herein refers to a binder that specifically binds to a target molecule, such as CCR8, in two different species. A CCR8 binder with cross-reactivity towards a non-human primate and human CCR8 specifically binds to both the human and said non-human primate forms of CCR8. As is known in the field, cross-reactive binders typically have a somewhat different affinity for target protein in both species. Preferably, the cross-reactive binder has a ratio of the dissociation constant for human CCR8 to the dissociation constant for the CCR8 in the other species in a range of 1000:1 to 1:1000; such as 500:1 to 1:500. In particular a range of 200:1 to 1:200, more in particular 100:1 to 1:100. Thus, in particular, the dissociation constant difference to human and e.g. macaque CCR8 is equal to or less than 100-fold.

As used herein, the term “non-human primate” comprises all species of primates, other than human beings. This includes for example monkeys from the genus Macaca (such as, in particular, cynomologus monkeys (Macaca fascicularis) and/or rhesus monkeys (Macaca mulatta)) and baboon (Papio ursinus) and/or African green monkeys (Chiorocebus aethiops).

“Specific binding”, “bind specifically”, and “specifically bind” is particularly understood to mean that the binder has a dissociation constant (K d) for the antigen of interest of less than about 10⁻⁶ M, 10⁻⁷ M, 10⁻⁶ M, 10⁻⁹ M, 10⁻¹⁰ M, 10⁻¹¹ M, 10⁻¹² M or 10⁻¹³ M. In a preferred embodiment, the dissociation constant is less than 10⁻⁸ M, for instance in the range of 10⁻⁹ M, 10⁻¹⁰ M, 10⁻¹¹ M, 10⁻¹² M or 10⁻¹³ M. Binder affinities towards membrane targets may be determined by a surface plasmon resonance based assay (such as the BIAcore assay as described in PCT Application Publication No. WO2005/012359) using viral like particles; cellular enzyme-linked immunoabsorbent assay (ELISA); and fluorescent activated cell sorting (FACS) read outs for example. A preferred method for determining apparent Kd or EC50 values is by using FACS at 21° C. with cells overexpressing hCCR8.

In a particular embodiment, the binding moiety of the hCCR8 binder is proteinaceous, more particularly a hCCR8 binding polypeptide. In a further embodiment, the binding moiety of the hCCR8 binder is antibody based or non-antibody based, preferably antibody based. Non-antibody based binders include, but are not limited to, affibodies, Kunitz domain peptides, monobodies (adnectins), anticalins, designed ankyrin repeat domains (DARPins), centyrins, fynomers, avimers; affilins; affitins, peptides and the like. In a particular embodiment, the hCCR8 binder of the invention binds to an extracellular part of hCCR8, in particular an extracellular part of hCCR8 expressed on regulatory T-cells. In a particular embodiment, the hCCR8 binder of the invention binds to an extracellular loop, especially extracellular loop 2 or extracellular loop 3 of hCCR8.

As described herein, the terms “antibody”, “antibody fragment” and “active antibody fragment” refer to a protein comprising an immunoglobulin (Ig) domain or an antigen-binding domain capable of specifically binding the antigen, in this case the hCCR8 protein. “Antibodies” can further be intact immunoglobulins derived from natural sources or from recombinant sources and can be immunoreactive portions of intact immunoglobulins. Antibodies may be multimers, such as tetramers, of immunoglobulin molecules. In a preferred embodiment, the binder comprises a hCCR8 binding moiety that is an antibody or active antibody fragment. In a further aspect of the invention, the binder is an antibody. In a further aspect of the invention the antibody is monoclonal. The antibody may additionally or alternatively be humanised or human. In a further aspect, the antibody is human, or in any case an antibody that has a format and features allowing its use and administration in human subjects. Antibodies may be derived from any species, including but not limited to mouse, rat, chicken, rabbit, goat, bovine, non-human primate, human, dromedary, camel, llama, alpaca, and shark.

The term “antigen-binding fragment” is intended to refer to an antigen-binding portion of said intact polyclonal or monoclonal antibodies that retains the ability to specifically bind to a target antigen or a single chain thereof, fusion proteins comprising an antibody, and any other modified configuration of the immunoglobulin molecule that comprises an antigen recognition site. The antigen-binding fragment comprises, but not limited to Fab; Fab′; F(ab′)2; a Fc fragment; a single domain antibody (sdAb or dAb) fragment. These fragments are derived from intact antibodies by using conventional methods in the art, for example by proteolytic cleavage with enzymes such as papain (to produce Fab fragments) or pepsin (to produce F(ab′)2 fragments). As used herein, antigen-binding fragment also refers to fusion proteins comprising heavy and/or light chain variable regions, such as single-chain variable fragments (scFv).

As used herein, the term “monoclonal antibody” refers to an antibody composition having a homogeneous antibody population. It is understood that monoclonal antibodies are highly specific, being directed against a single antigenic site. Furthermore, in contrast to conventional antibody (polyclonal) preparations which typically include different antibodies directed against different determinants (epitopes), each monoclonal antibody is directed against a single determinant on the antigen. The binders of the invention preferably comprise a monoclonal antibody moiety that binds to hCCR8.

In one aspect of the invention, the binder comprises an active antibody fragment. The term “active antibody fragment” refers to a portion of any antibody or antibody-like structure that by itself has high affinity for an antigenic determinant, or epitope, and contains one or more antigen-binding sites, e.g. complementary-determining-regions (CDRs), accounting for such specificity. Non-limiting examples include immunoglobulin domains, Fab, F(ab)′2, scFv, heavy-light chain dimers, immunoglobulin single variable domains, single domain antibodies (sdAb or dAb), Nanobodies®, and single chain structures, such as complete light chain or complete heavy chain, as well as antibody constant domains that have been engineered to bind to an antigen. An additional requirement for the “activity” of said fragments in the light of the present invention is that said fragments are capable of binding hCCR8. The term “immunoglobulin (Ig) domain” or more specifically “immunoglobulin variable domain” (abbreviated as “IVD”) means an immunoglobulin domain essentially consisting of framework regions interrupted by complementary determining regions. Typically, immunoglobulin domains consist essentially of four “framework regions” which are referred in the art and below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred in the art and herein below as “complementarity determining region 1” or “CDR1”; as “complementarity determining region 2” or “CDR2”; and as “complementarity determining region 3” or “CDR3”, respectively. Thus the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site. Typically, in conventional immunoglobulins, a heavy chain variable domain (VH) and a light chain variable domain (VL) interact to form an antigen binding site. In this case the complementary determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as IgG, IgM, IgA, IgD or IgE molecule; known in the art) or of a Fab fragment, a F(ab′)2 fragment, an Fv fragment such as a disulphide linked Fv or scFv fragment, or a diabody (all known in the art) derived from such conventional 4-chain antibody, with binding to the respective epitope of an antigen by a pair of (associated) immunoglobulin domains such as light and heavy chain variable domains, i.e., by a VH-VL pair of immunoglobulin domains, which jointly bind to an epitope of the respective antigen. A single-domain antibody (sdAb) as used herein, refers to a protein with an amino acid sequence comprising 4 framework regions (FR) and 3 complementarity determining regions (CDRs) according to the format FR1-CDR1-FR2-CDR2-FR3-CDR3-FR4. Single-domain antibodies of this invention are equivalent to “immunoglobulin single variable domains” (abbreviated as “ISVD”) and refers to molecules wherein the antigen binding site is present on, and formed by, a single immunoglobulin domain. This sets single-domain antibodies apart from “conventional” antibodies or their fragments, wherein two immunoglobulin domains, in particular two variable domains interact to form an antigen binding site. The binding site of a single-domain antibody is formed by a single VH/VHH or VL domain. Hence, the antigen binding site of a single-domain antibody is formed by no more than 3 CDRs. As such a single domain may be a light chain variable domain sequence. (e.g. a VL-sequence) or a suitable fragment thereof; or a heavy chain variable domain sequence (e.g. a VH-sequence or VHH sequence) or a suitable fragment thereof; as long as it is capable of forming a single antigen binding unit (i.e., a functional antigen binding unit that essentially consists of a single variable domain, such that the single antigen binding domain does not need to interact with another variable domain to form a functional antigen binding unit).

Thus, in one embodiment, the hCCR8 binder as detailed above, comprises a single-domain antibody moiety.

In particular, the single-domain antibody may be a Nanobody® (as defined herein) or a suitable fragment thereof (Note: Nanobody®, Nanobodies® and Nanoclone® are registered trademarks of Ablynx N. V., a Sanofi Company). For general description of Nanobodies® reference is made to the further description below, and described in the prior art such as e.g. WO2008/020079. “VHH domains”, also known as VHHs, VHH antibody fragments and VHH antibodies, have originally been described as the antigen binding immunoglobulin (Ig) (variable) domain of “heavy chain antibodies” (i.e. of “antibodies devoid of light chains”; see e.g. Hamers-Casterman et al., Nature 363:446-8 (1993)). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobodies®, reference is made to the review article by Muyldermans (Reviews in Molecular Biotechnology 74: 277-302, 2001), as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N. V. and Ablynx N. V.; WO 01/90190 by the National Research Council of Canada; WO 03/025020 (=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N. V. and the further published patent applications by Ablynx N. V. As described in these references, Nanobody® (in particular VHH sequences and partially humanized Nanobody®) can in particular be characterized by the presence of one or more “Hallmark residues” in one or more of the framework sequences. A further description of the Nanobody®, including humanization and/or camelization of Nanobody, as well as other modifications, parts or fragments, derivatives or “Nanobody fusions”, multivalent or multispecific constructs (including some non-limiting examples of linker sequences) and different modifications to increase the half-life of the Nanobody® and their preparations can be found e.g. in WO 08/101985 and WO 08/142164. VHHs and Nanobodies® are among the smallest antigen binding fragment that completely retains the binding affinity and specificity of a full-length antibody (see e.g. Greenberg et al., Nature 374:168-73 (1995); Hassanzadeh-Ghassabeh et al., Nanomedicine (Lond), 8:1013-26 (2013)).

The binders of the present invention may be monospecific, bispecific, or multispecific. “Multispecific binders” may be specific for different epitopes of one target antigen or polypeptide, or may contain antigen-binding domains specific for more than one target antigen or polypeptide (Kufer et al. Trends Biotechnol 22:238-44 (2004)).

In one aspect of the invention, the binder is a monospecific binder. As discussed further below, in an alternative aspect the binder is a bispecific binder.

As used herein, “bispecific binder” refers to a binder having the capacity to bind two distinct epitopes either on a single antigen or polypeptide, or on two different antigens or polypeptides.

Bispecific binders of the present invention as discussed herein can be produced via biological methods, such as somatic hybridization; or genetic methods, such as the expression of a non-native DNA sequence encoding the desired binder structure in a cell line or in an organism; chemical methods (e.g. by chemical coupling, genetic fusion, noncovalent associated or otherwise to one or more molecular entities, such as another binder of fragment thereof); or combination thereof.

The technologies and products that allow producing monospecific or bispecific binders are known in the art, as extensively reviewed in the literature, also with respect to alternative formats, binder-drug conjugates, binder design methods, in vitro screening methods, constant regions, post-translational and chemical modifications, improved feature for triggering cancer cell death such as Fc domain engineering (Tiller K and Tessier P, Annu Rev Biomed Eng. 17:191-216 (2015); Speiss C et al., Molecular Immunology 67:95-106 (2015); Weiner G, Nat Rev Cancer, 15:361-370 (2015); Fan G et al., J Hematol Oncol 8:130 (2015)).

The hCCR8 binders of the invention may be blocking or non-blocking binders. In a particular embodiment, the hCCR8 binders of the invention are blocking binders, in the art also described as neutralizing binders. In a particular embodiment, the hCCR8 binder of the invention inhibits the binding of hCCL1 and/or other hCCR8 ligands to hCCR8.

As used herein, “epitope” or “antigenic determinant” refers to a site on an antigen to which a binder, such as an antibody, binds. As is well known in the art, epitopes can be formed both from contiguous amino acids (linear epitope) or non-contiguous amino acids juxtaposed by tertiary folding of a protein (conformational epitopes). Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes are well known in the art and include, for example, x-ray crystallography and 2-D nuclear magnetic resonance. See, for example, Epitope Mapping Protocols in Methods in Molecular Biology, Vol. 66, Glenn E. Morris, Ed (1996).

As used herein, the term “sequence identity” means that two polypeptide or polynucleotide sequences are identical (i.e. on an amino acid-by-amino acid, or on a nucleotide-by-nucleotide basis, respectively) over a window of comparison. The term “percentage of sequence identity” is calculated by comparing two optimally aligned sequences over the window of comparison, determining the number of positions at which the identical amino acid or nucleic acid base, whichever relevant, occurs in both sequences to yield the number of matched positions, dividing the number of matched positions by the total number of positions in the window of comparison (i.e. the window size), and multiplying the result by 100 to yield the percentage of sequence identity.

As used herein, the term “substantially identical” or “substantial identity” denotes a characteristic of a polypeptide or polynucleotide sequence, wherein the polypeptide or polynucleotide comprises a sequence that has at least 80% sequence identity, preferably at least 85% sequence identify, more preferably 90% sequence identity, still more preferably 95% sequence identity, yet more preferably 99% sequence identity as compared to a reference sequence, wherein the percentage of sequence identify is calculated by aligning the reference sequence to the polypeptide or polynucleotide sequence which may include deletions or additions which in total amount 20% or less of the reference sequence over the window of comparison. The reference sequence may be a subset of a larger sequence. Optimal alignment of sequences may be carried out by conventional software or methods known by those of ordinary skill in the art.

As used herein, the term “corresponds to” or “corresponding to” is intended to mean that a polypeptide or a polynucleotide sequence is identical or similar to all or a portion of a reference polypeptide or polynucleotide sequence. In contradistinction, the term “complementary to” as used herein in the relation to a polypeptide or polynucleotide sequence is intended to mean that the complementary sequence is homologous to all or a portion of a reference polypeptide or polynucleotide sequence. For illustration, the nucleotide sequence “TATAC” corresponds to a reference sequence “TATAC” and is complementary to a reference sequence “GTATA”.

In one embodiment of the present invention, the hCC8 binder, as detailed above, comprises a single-domain antibody moiety which comprises at least one complementarity determining region (CDR) of a single-domain antibody moiety as described herein, or an amino acid sequence having at least 80% amino acid identity the said CDR sequences, or an amino acid sequence having 3, 2, or 1 amino acid sequence difference with said CDR sequences. It is understood that the CDRs and the locations thereof in the sequence of said single-domain antibody moiety can be readily identified by conventional methods known by those of ordinary skill in the art, such as but not limited to, KABAT system (Kabat), Chothia, AHo or international ImMunoGeneTics information system (IMGT). The preferred method for determining CDR sequences is the IMGT method (Lefranc, M.-P. et al., 2009, Nucleic Acids Research, D1006-1012, http://www.imgt.org).

As will be described in the examples below, a specific and preferred hCCR8 binder according to the invention comprises a single-domain antibody moiety corresponding to SEQ ID NO: 21-25 or 35-38. FIG. 6 presents a schematic representation of the amino acid sequence of SEQ ID NOs: 35-38, wherein CDRs are identified using the IMGT method (bold) or the Kabat method (underlined).

Accordingly, using the IMGT method, the CDRs as identified within the single-domain antibody moiety, as defined above, correspond to:

SEQ ID NO: 1
(GFSFSSFA)
SEQ ID NO: 2
(GRAFSSYN)
SEQ ID NO: 3
(RSIYNVLA)
SEQ ID NO: 4
(RSIYNVRA)
SEQ ID NO: 5
(GSTFSIKS)
SEQ ID NO: 6
(ITTTGAT)
SEQ ID NO: 7
(ISSSGT)
SEQ ID NO: 8
(VWSSGNT)
SEQ ID NO: 9
(ITRGAGI)
SEQ ID NO: 10
(NARARLWSVFDY)
SEQ ID NO: 11
(NAKTVTRTGGVITGSRIEYDY)
SEQ ID NO: 12
(YSKSLIGTSRYEI)
SEQ ID NO: 13
(VKTIKRTGGILTGSKIEYDY)
SEQ ID NO: 26
(RSIYNVMA)
SEQ ID NO: 27
(NARARLSSVFDY)
SEQ ID NO: 28
(NVKTIKRTGGIMTGSKIEYDY)

Alternatively, the CDRs as identified within the single-domain antibody moiety, as defined above, correspond to:

SEQ ID NO: 39
(GFSFSSFALS)
SEQ ID NO: 40
(GRAFSSYNMG)
SEQ ID NO: 41
(RSIYNVMAMG)
SEQ ID NO: 42
(GSTFSIKSIG)
SEQ ID NO: 43
(RITTTGATNYADSVKG)
SEQ ID NO: 44
(TISSSGTNYANSVKG)
SEQ ID NO: 45
(VVWSSGNTNYADSVKG)
SEQ ID NO: 46
(DITRGAGINYADSVKG)
SEQ ID NO: 47
(RSIYNVLAMG)
SEQ ID NO: 48
(RSIYNVRAMG)
SEQ ID NO: 49
(TISSSGTNYADSVKG)
SEQ ID NO: 10
(NARARLWSVFDY)
SEQ ID NO: 11
(NAKTVTRTGGVITGSRIEYDY)
SEQ ID NO: 12
(YSKSLIGTSRYEI)
SEQ ID NO: 13
(VKTIKRTGGILTGSKIEYDY)

Furthermore, using the Kabat method (Kabat E. A. et al., 1991, Sequences of Proteins of Immunological Interest, Fifth Edition, NIH Publication No. 91-3242), the CDRs as identified within the single-domain antibody moiety, as defined above, correspond to:

SEQ ID NO: 81
(SFALS)
SEQ ID NO: 82
(SYNMG)
SEQ ID NO: 83
(VMAMG)
SEQ ID NO: 84
(IKSIG)
SEQ ID NO: 85
(VLAMG)
SEQ ID NO: 86
(VRAMG)
SEQ ID NO: 43
(RITTTGATNYADSVKG)
SEQ ID NO: 44
(TISSSGTNYANSVKG)
SEQ ID NO: 45
(VVWSSGNTNYADSVKG)
SEQ ID NO: 46
(DITRGAGINYADSVKG)
SEQ ID NO: 49
(TISSSGTNYADSVKG)
SEQ ID NO: 87
(RARLSSVFDY)
SEQ ID NO: 88
(RARLWSVFDY)
SEQ ID NO: 89
(KTVTRTGGVITGSRIEYDY)
SEQ ID NO: 90
(KSLIGTSRYEI)
SEQ ID NO: 91
(KTIKRTGGIMTGSKIEYDY)
SEQ ID NO: 92
(KTIKRTGGILTGSKIEYDY)

Thus, the single-domain antibody moiety, as detailed above comprises at least one, preferably at least two and most preferably three CDR(s) selected from the group consisting of SEQ ID NO: 1-13 and SEQ ID NO: 26-28, or at least one, preferably at least two and most preferably three amino acid sequence(s) having at least 80% amino acid identity to said CDR sequences, or at least one, preferably at least two and most preferably three amino acid sequence(s) having 3, 2, or 1 amino acid sequence difference with said CDR sequences.

Preferably, the single-domain antibody moiety, as detailed above comprises a CDR3 which is selected from the group consisting of (a) the amino acid sequence of NARARLWSVFDY (SEQ ID NO: 10), (b) the amino acid sequence of NAKTVTRTGGVITGSRIEYDY (SEQ ID NO: 11), (c) the amino acid sequence of YSKSLIGTSRYEI (SEQ ID NO: 12), (d) the amino acid sequence of NVKTIKRTGGILTGSKIEYDY (SEQ ID NO: 13), (e) the amino acid sequence of NARARLSSVFDY (SEQ ID NO: 27), (f) the amino acid sequence of NVKTIKRTGGIMTGSKIEYDY (SEQ ID NO: 28), (g) amino acid sequences having at least 80% amino acid sequence identity with SEQ ID NO: 10, 11, 12, 13, 27 or 28, and (h) amino acid sequences having 3, 2 or 1 amino acid different from SEQ ID NO: 10, 11, 12, 13, 27 or 28. More preferably, CDR3 corresponds to SEQ ID NO: 10, 11, 12, 13, 27 or 28.

In a preferred embodiment, CDR1 is selected from the group consisting of (a) the amino acid sequence of GFSFSSFA (SEQ ID NO: 1), (b) the amino acid sequence of GRAFSSYN (SEQ ID NO: 2), (c) the amino acid sequence of RSIYNVLA (SEQ ID NO: 3), (d) the amino acid sequence of RSIYNVRA (SEQ ID NO: 4), (e) the amino acid sequence of GSTFSIKS (SEQ ID NO: 5), (f) the amino acid sequence of RSIYNVMA (SEQ ID NO: 26), (g) amino acid sequences having at least 80% amino acid identity with SEQ ID NO: 1, 2, 3, 4, 5 or 26, and (h) amino acid sequences having 3, 2, 1 amino acid difference from SEQ ID NO: 1, 2, 3, 4, 5 or 26, and/or CDR2 is selected from the group consisting of (a) the amino acid sequence of ITTTGAT (SEQ ID NO: 6), (b) the amino acid sequence of ISSSGT (SEQ ID NO: 7), (c) the amino acid sequence of VWSSGNT (SEQ ID NO: 8), (d) the amino acid sequence of ITRGAGI (SEQ ID NO: 9), (e) amino acid sequences having at least 80% amino acid identity with SEQ ID NO: 6, 7, 8 or 9, and (f) amino acid sequences having 3, 2, 1 amino acid difference from SEQ ID NO: 6, 7, 8 or 9.

In another particular embodiment, the present invention provides a hCCR8 binder comprising a combination of CDR1, CDR2, and CDR3 as described herein, including the allowable variation described for these CDR regions. In another particular embodiment, the binder of the invention comprises at least one CDR region of the single-domain antibody moieties as described herein. In a further embodiment, the binder of the invention comprises at least one CDR region of a single-domain antibody moiety having the amino acid sequence of SEQ ID NO: 35, 36, 37 or 38. In an even further embodiment, the binder of the invention comprises the three CDR regions of a single-domain antibody moiety having the amino acid sequence of SEQ ID NO: 35, 36, 37 or 38.

In a more preferred embodiment, the single-domain antibody moiety, as detailed above comprises three CDRs having the sequence of SEQ ID NO: 1, 6 and 27, or comprises three CDRs having the sequence of SEQ ID NO: 2, 7 and 11, or comprises three CDRs having the sequence of SEQ ID NO: 8, 12 and 26, or comprises three CDRs having the sequence of SEQ ID NO: 5, 9 and 28.

In another embodiment of the invention, the single-domain antibody moiety, as detailed above, further comprises a sequence having at least 85, 90, 95, 98 or 99% sequence identify to at least one framework region (FR) of a single-domain antibody moiety described herein. In another embodiment of the invention, the single-domain antibody moiety, as detailed above, further comprises a sequence having at least 85, 90, 95, 98 or 99% sequence identify to the four framework regions (FR) of a single-domain antibody moiety described herein. It is understood that the method used for determining the FRs of said single-domain antibody moiety is the same as that used for identifying the CDRs.

Accordingly, using the IMGT method, the FRs as identified within the single-domain antibody moiety, as defined above, correspond to:

SEQ ID NO: 14
(DVQLVESGGGLVQPGGSLRLSCAAS)
SEQ ID NO: 15
(MGWYRQAPGKERELVAV)
SEQ ID NO: 16
(LSWYRQAPGKERELVAR)
SEQ ID NO: 17
(MGWFRQAPGKEREFVAT)
SEQ ID NO: 18
(GWYRQAPGKQRELVAD)
SEQ ID NO: 19
(NYADSVKGRFTISRDNAKNTVYLQMNSLRPEDTAVYYC)
SEQ ID NO: 20
(WGQGTLVTVSS)
SEQ ID NO: 29
(EVQLVESGGGLVQPGGSLRLSCAAS)
SEQ ID NO: 30
(MGWFRQAPGKERDFVAT)
SEQ ID NO: 31
(NYADSVKGRFTISRDNAKNMGYLQMNSLRPEDTAVYYC)
SEQ ID NO: 32
(NYANSVKGRFTISRDNAKNTVYLQMNSLRPEDTAVYYC)
SEQ ID NO: 33
(NYADSVKGRFTVSRDNAKNTVYLQMNSLRPEDTAVYYC)
SEQ ID NO: 34
(NYADSVKGRFTISRDNTKNTMYLQMNSLRPEDTAVYYC)

Thus, the single-domain antibody moiety, as detailed above comprises at least one, preferably at least two, more preferably at least three and most preferably four amino acid sequences having at least 85%, preferably 90%, more preferably 95% sequence identity to the sequences selected from the group consisting of SEQ ID NO: 14-20 and 29-34.

Preferably, said single-domain antibody moiety, as detailed above comprises four framework regions (FRs) according to the format FR1-CDR1-FR2-CDR2-FR3-CDR3-FR4, wherein FR1 has at least 85%, preferably 90%, more preferably 95% sequence identity to the sequence of SEQ ID NO: 14 or 29, FR2 has at least 85%, preferably 90%, more preferably 95% sequence identity to the sequence of SEQ ID NO: 15-18 or 30, FR3 has at least 85%, preferably 90%, more preferably 95% sequence identity to the sequence of SEQ ID NO: 19 or 31-34, and FR4 has at least 85%, preferably 90%, more preferably 95% sequence identity to the sequence of SEQ ID NO: 20.

In another particular embodiment, the binder of the present invention comprises an antibody or antigen-binding fragment thereof that comprises an amino acid sequence of SEQ ID NO: 35, 36, 37 or 38, or an amino acid sequence having 85%, 90% or 95% sequence identity thereto; wherein the binder comprises a CDR1 of SEQ ID NO: 1, 2, 5 or 26, a CDR2 of SEQ ID NO: 6, 7, 8 or 9, and a CDR3 of SEQ ID NO: 11, 12, 27 or 28.

In a particular embodiment, the binder of the invention comprises the amino acid sequences corresponding to SEQ ID NO: 35. In another particular embodiment, the binder of the invention comprises the amino acid sequences corresponding to SEQ ID NO: 36. In yet another particular embodiment, the binder of the invention comprises the amino acid sequences corresponding to SEQ ID NO: 37. In yet another particular embodiment, the binder of the invention comprises the amino acid sequences corresponding to SEQ ID NO: 38.

Sequence Optimized hCCR8 Binders

As used herein, the term “humanized binder” refers to a binder produced by molecular modelling techniques to identify an optimal combination of human and non-human (such as mouse or rabbits) binder sequences, that is, a combination in which the human content of the binder is maximized while causing little or no loss of the binding affinity attributable to the variable region of the non-human antibody. For example, a humanized antibody, also known as a chimeric antibody comprises the amino acid sequence of a human framework region and of a constant region from a human antibody to “humanize” or render non-immunogenic the complementarity determining regions (CDRs) from a non-human antibody.

As used herein, the term “human binder” means a binder having an amino acid sequence corresponding to that of a binder that can be produced by a human and/or which has been made using any of the techniques for making human antibodies known to a skilled person in the art or disclosed herein. It is also understood that the term “human antibody” encompasses antibodies comprising at least one human heavy chain polypeptide or at least one human light chain polypeptide. One such example is an antibody comprising murine light chain and human heavy chain polypeptides.

As for full-size antibodies, single variable domains such as VHHs and Nanobodies® can be subjected to sequence optimization, such as humanization, i.e. increase the degree of sequence identity with the closest human germline sequence, and other optimization techniques, such as to improve physicochemical or other properties of the binders. In particular, humanized immunoglobulin single variable domains, such as VHHs and Nanobodies® may be single-domain antibodies in which at least one single amino acid residue is present (and in particular, at least one framework residue) that is and/or that corresponds to a humanizing substitution (as defined further herein).

Humanized single-domain antibodies, in particular VHHs and Nanobodies®, may have several advantages, such as a reduced immunogenicity, compared to the corresponding naturally occurring VHH domains. By humanized is meant mutated so that immunogenicity upon administration in human patients is minor or non-existent. The humanizing substitutions should be chosen such that the resulting humanized amino acid sequence and/or VHH still retains the favourable properties of the VHH, such as the antigen-binding capacity.

In one particular embodiment, the hCCR8 binder, as described above, is an optimized hCCR8 binder.

Preferably, the optimized hCCR8 binder comprises a single-domain antibody moiety as described above. More preferably, the single-domain antibody has been optimized by introducing mutations, in particular substitutions, for example at any one of positions of 1, 77, 78 and/or 103 of SEQ ID NO: 35, or at any one of positions of 1, 46 and/or 60 of SEQ ID NO: 36, or at any one of positions of 1, 32 and/or 69 of SEQ ID NO: 37, or at any one of positions of 1, 74, 78 and/or 107 of SEQ ID NO: 38.

These residues have been highlighted in FIG. 8. It was found that these sequence optimization mutations did not substantially affect target binding parameters. Interestingly, it was found that the E1D mutation in all of SEQ ID NO: 35-38 eliminated pyroglutamatic acid formation. Furthermore, substituting a methionine residue (M) by either a leucine (L) or an arginine (R) at position 32 in SEQ ID NO: 37 improved expression levels of VHH-114 and VHH-115 in E. coli as compared to the controls VHH-56(E1D) and VHH-56(E1D,V691). In addition, it was found that the combination of the substitutions M32L or M32R with V691 in SEQ ID NO: 37 improved thermal stability compared with the parental SEQ ID NO: 37 sequence. Also, in SEQ ID NO: 35, the combined substitutions G78V and M77T improved thermal stability of the binders compared to the parental SEQ ID NO: 35 sequence, while the additional introduction of substitution S103W yet further improved thermal stability. The threonine (T) to alanine (A) substitution at position 74 in SEQ ID NO: 38 also improved thermal stability. Interestingly, substituting a methionine residue (M) by a leucine (L) at position 107 of SEQ ID NO: 38 considerably reduced levels of forced oxidation, as shown in the examples below. Substitution of an aspartic acid (D) by a glutamic acid (E) at position 46 of SEQ ID NO: 36 also improved the thermal stability. The other amino acid substitutions described above were mainly performed to humanize further the binders.

Therefore, in a particular embodiment, the present invention provides a binder comprising the amino acid sequence of SEQ ID NO: 35, optionally comprising one or more of the substitutions E1D, M77T, G78V, and S103W. In a further embodiment, a binder comprising the amino acid sequence of SEQ ID NO: 36 comprising one or more of the substitutions E1D, D46E, and N60D. In yet a further embodiment, a binder comprising the amino acid sequence of SEQ ID NO: 37 comprising one or more of the substitutions E1D, M32L, and V691. In yet a further embodiment, a binder comprising the amino acid sequence of SEQ ID NO: 37 comprising one or more of the substitutions E1D, M32R, and V691. In another embodiment, a binder comprising the amino acid sequence of SEQ ID NO: 38 comprising one or more of the substitutions E1D, T74A, M78V, and M107L. In a further embodiment, the present invention provides a binder comprising the amino acid sequence of SEQ ID NO: 38, comprising the substitution M107L and optionally one or more substitutions of E1D, T74A, and M78V.

In another embodiment, the binder of the invention comprises at least one CDR region of a single-domain antibody moiety having the amino acid sequence of SEQ ID NO: 21. In an even further embodiment, the binder of the invention comprises the three CDR regions of a single-domain antibody moiety having the amino acid sequence of SEQ ID NO: 21. In yet another embodiment, the binder of the invention comprises at least one CDR region of a single-domain antibody moiety having the amino acid sequence of SEQ ID NO: 22. In an even further embodiment, the binder of the invention comprises the three CDR regions of a single-domain antibody moiety having the amino acid sequence of SEQ ID NO: 22. In yet another embodiment, the binder of the invention comprises at least one CDR region of a single-domain antibody moiety having the amino acid sequence of SEQ ID NO: 23. In an even further embodiment, the binder of the invention comprises the three CDR regions of a single-domain antibody moiety having the amino acid sequence of SEQ ID NO: 23. In yet another embodiment, the binder of the invention comprises at least one CDR region of a single-domain antibody moiety having the amino acid sequence of SEQ ID NO: 24. In an even further embodiment, the binder of the invention comprises the three CDR regions of a single-domain antibody moiety having the amino acid sequence of SEQ ID NO: 24. In yet another embodiment, the binder of the invention comprises at least one CDR region of a single-domain antibody moiety having the amino acid sequence of SEQ ID NO: 25. In an even further embodiment, the binder of the invention comprises the three CDR regions of a single-domain antibody moiety having the amino acid sequence of SEQ ID NO: 25.

However, the amino acid sequences and/or single-domain antibody of the invention may be suitably optimized at any position and in particular at any framework residue(s), such as at one or more Hallmark residues (as defined above) or at one or more other framework residues (i.e. non-Hallmark residues) or any suitable combination thereof. Depending on the host organism used to express the amino acid sequence, single-domain antibody or polypeptide of the invention, such deletions and/or substitutions may also be designed in such a way that one or more sites for posttranslational modification (such as one or more glycosylation sites) are removed, as will be within the ability of the person skilled in the art. Alternatively, substitutions or insertions may be designed so as to introduce one or more sites for attachment of functional groups (as described herein), for example to allow site-specific pegylation.

In one embodiment of the present invention, the optimized hCC8 binder, as detailed above, comprises a single-domain antibody moiety which comprises at least one complementarity determining region (CDR) of a single-domain antibody moiety as described herein, or an amino acid sequence having at least 80% amino acid identity the said CDR sequences, or an amino acid sequence having 3, 2, or 1 amino acid sequence difference with said CDR sequences. It is understood that the CDRs and the locations thereof in the sequence of said single-domain antibody moiety can be readily identified by conventional methods known by those of ordinary skill in the art, such as but not limited to, KABAT system (Kabat), Chothia, AHo or international ImMunoGeneTics information system (IMGT). The preferred method for determining CDR sequences is the IMGT method (Lefranc, M.-P. et al., 2009, Nucleic Acids Research, D1006-1012, http://www.imgt.org).

As will be described in the examples below, five specific optimized hCCR8 binders according to the invention comprise a single-domain antibody moiety corresponding to SEQ ID NO: 21, 22, 23, 24 or 25.

Accordingly, using the IMGT method, the CDRs as identified within the single-domain antibody moiety, as defined above, correspond to:

SEQ ID NO: 1
(GFSFSSFA)
SEQ ID NO: 2
(GRAFSSYN)
SEQ ID NO: 3
(RSIYNVLA)
SEQ ID NO: 4
(RSIYNVRA)
SEQ ID NO: 5
(GSTFSIKS)
SEQ ID NO: 6
(ITTTGAT)
SEQ ID NO: 7
(ISSSGT)
SEQ ID NO: 8
(VWSSGNT)
SEQ ID NO: 9
(ITRGAGI)
SEQ ID NO: 10
(NARARLWSVFDY)
SEQ ID NO: 11
(NAKTVTRTGGVITGSRIEYDY)
SEQ ID NO: 12
(YSKSLIGTSRYEI)
SEQ ID NO: 13
(VKTIKRTGGILTGSKIEYDY)

Alternatively, the CDRs as identified within the single-domain antibody moiety, as defined above, correspond to:

SEQ ID NO: 39
(GFSFSSFALS)
SEQ ID NO: 40
(GRAFSSYNMG)
SEQ ID NO: 42
(GSTFSIKSIG)
SEQ ID NO: 43
(RITTTGATNYADSVKG)
SEQ ID NO: 45
(VVWSSGNTNYADSVKG)
SEQ ID NO: 46
(DITRGAGINYADSVKG)
SEQ ID NO: 47
(RSIYNVLAMG)
SEQ ID NO: 48
(RSIYNVRAMG)
SEQ ID NO: 49
(TISSSGTNYADSVKG)
SEQ ID NO: 10
(NARARLWSVFDY)
SEQ ID NO: 11
(NAKTVTRTGGVITGSRIEYDY)
SEQ ID NO: 12
(YSKSLIGTSRYEI)
SEQ ID NO: 13
(VKTIKRTGGILTGSKIEYDY)

Furthermore, using the Kabat method, the CDRs as identified within the single-domain antibody moiety, as defined above, correspond to:

SEQ ID NO: 81
(SFALS)
SEQ ID NO: 82
(SYNMG)
SEQ ID NO: 84
(IKSIG)
SEQ ID NO: 85
(VLAMG)
SEQ ID NO: 86
(VRAMG)
SEQ ID NO: 43
(RITTTGATNYADSVKG)
SEQ ID NO: 45
(VVWSSGNTNYADSVKG)
SEQ ID NO: 46
(DITRGAGINYADSVKG)
SEQ ID NO: 49
(TISSSGTNYADSVKG)
SEQ ID NO: 88
(RARLWSVFDY)
SEQ ID NO: 89
(KTVTRTGGVITGSRIEYDY)
SEQ ID NO: 90
(KSLIGTSRYEI)
SEQ ID NO: 92
(KTIKRTGGILTGSKIEYDY)

Thus, the single-domain antibody moiety, as detailed above comprises at least one, preferably at least two and most preferably three CDR(s) selected from the group consisting of SEQ ID NO: 1-13, or at least one, preferably at least two and most preferably three amino acid sequence(s) having at least 80% amino acid identity the said CDR sequences, or at least one, preferably at least two and most preferably three amino acid sequence(s) having 3, 2, or 1 amino acid sequence difference with said CDR sequences.

Preferably, the single-domain antibody moiety, as detailed above comprises a CDR3 which is selected from the group consisting of (a) the amino acid sequence of NARARLWSVFDY (SEQ ID NO: 10), (b) the amino acid sequence of NAKTVTRTGGVITGSRIEYDY (SEQ ID NO: 11), (c) the amino acid sequence of YSKSLIGTSRYEI (SEQ ID NO: 12), (d) the amino acid sequence of NVKTIKRTGGILTGSKIEYDY (SEQ ID NO: 13), (e) amino acid sequences having at least 80% amino acid sequence identity with SEQ ID NO: 10, 11, 12 or 13, and (f) amino acid sequences having 3, 2 or 1 amino acid different from SEQ ID NO: 10, 11, 12 or 13. More preferably, CDR3 corresponds to SEQ ID NO: 10, 11, 12 or 13.

In a preferred embodiment CDR1 is selected from the group consisting of (a) the amino acid sequence of GFSFSSFA (SEQ ID NO: 1), (b) the amino acid sequence of GRAFSSYN (SEQ ID NO: 2), (c) the amino acid sequence of RSIYNVLA (SEQ ID NO: 3), (d) the amino acid sequence of RSIYNVRA (SEQ ID NO: 4), (e) the amino acid sequence of GSTFSIKS (SEQ ID NO: 5), (f) amino acid sequences having at least 80% amino acid identity with SEQ ID NO: 1, 2, 3, 4 or 5, and (g) amino acid sequences having 3, 2, 1 amino acid difference from SEQ ID NO: 1, 2, 3, 4 or 5, and CDR2 is selected from the group consisting of (a) the amino acid sequence of the amino acid sequence of ITTTGAT (SEQ ID NO: 6), (b) the amino acid sequence of ISSSGT (SEQ ID NO: 7), (c) the amino acid sequence of VWSSGNT (SEQ ID NO: 8), (d) the amino acid sequence of ITRGAGI (SEQ ID NO: 9), (e) amino acid sequences having at least 80% amino acid identity with SEQ ID NO: 6, 7, 8 or 9, and (f) amino acid sequences having 3, 2, 1 amino acid difference from SEQ ID NO: 6, 7, 8 or 9.

In a more preferred embodiment, the single-domain antibody moiety, as detailed above comprises three CDRs having the sequences of SEQ ID NO: 1, 6 and 10, or having the sequences of SEQ ID NO: 2, 7 and 11, or having the sequences of SEQ ID NO: 3, 8 and 12, or having the sequences of SEQ ID NO: 4, 8 and 12, or having the sequences of SEQ ID NO: 5, 9 and 13.

In another embodiment of the invention, the single-domain antibody moiety, as detailed above, further comprises a sequence having at least 85, 90, 95, 98 or 99% sequence identify to at least one framework region (FR) of a single-domain antibody moiety described herein. It is understood that the method used for determining the FRs of said single-domain antibody moiety is the same as that used for identifying the CDRs.

Accordingly, using the IMGT method, the FRs as identified within the single-domain antibody moiety, as defined above, correspond to:

SEQ ID NO: 14
(DVQLVESGGGLVQPGGSLRLSCAAS)
SEQ ID NO: 15
(MGWYRQAPGKERELVAV)
SEQ ID NO: 16
(LSWYRQAPGKERELVAR)
SEQ ID NO: 17
(MGWFRQAPGKEREFVAT)
SEQ ID NO: 18
(GWYRQAPGKQRELVAD)
SEQ ID NO: 19
(NYADSVKGRFTISRDNAKNTVYLQMNSLRPEDTAVYYC)
SEQ ID NO: 20
(WGQGTLVTVSS)

Thus, the single-domain antibody moiety, as detailed above comprises at least one, preferably at least two, more preferably at least three and most preferably four amino acid sequences having at least 85%, preferably 90%, more preferably 95% sequence identity to the sequences selected from the group consisting of SEQ ID NO: 14-22.

Preferably, said single-domain antibody moiety, as detailed above four framework regions (FRs) according to the format FR1-CDR1-FR2-CDR2-FR3-CDR3-FR4, wherein FR1 has at least 85%, preferably 90%, more preferably 95% sequence identity to the sequence of SEQ ID NO: 14, FR2 has at least 85%, preferably 90%, more preferably 95% sequence identity to the sequence of SEQ ID NO: 15, 16, 17 or 18, FR3 has at least 85%, preferably 90%, more preferably 95% sequence identity to the sequence of SEQ ID NO: 19, and FR4 has at least 85%, preferably 90%, more preferably 95% sequence identity to the sequence of SEQ ID NO: 20.

More preferably, said single-domain antibody moiety, as detailed above comprises the amino acid sequences corresponding to SEQ ID NO: 21, 22, 23, 24 or 25. In a particular embodiment, the binder of the invention comprises the amino acid sequence of SEQ ID NO: 21. In another particular embodiment, the binder of the invention comprises the amino acid sequence of SEQ ID NO: 22. In yet another particular embodiment, the binder of the invention comprises the amino acid sequence of SEQ ID NO: 23. In still another particular embodiment, the binder of the invention comprises the amino acid sequence of SEQ ID NO: 24. In another particular embodiment, the binder of the invention comprises the amino acid sequence of SEQ ID NO: 25.

In another particular embodiment, the binder of the present invention comprises an antibody or antigen-binding fragment thereof that comprises an amino acid sequence of SEQ ID NO: 21 or an amino acid sequence having 85%, 90% or 95% sequence identity thereto, wherein the binder comprises a CDR1 of SEQ ID NO: 1, a CDR2 of SEQ ID NO: 6, and a CDR3 of SEQ ID NO: 10.

In yet another particular embodiment, the binder of the present invention comprises an antibody or antigen-binding fragment thereof that comprises an amino acid sequence of SEQ ID NO: 22 or an amino acid sequence having 85%, 90% or 95% sequence identity thereto, wherein the binder comprises a CDR1 of SEQ ID NO: 2, a CDR2 of SEQ ID NO: 7, and a CDR3 of SEQ ID NO: 11.

In still another particular embodiment, the binder of the present invention comprises an antibody or antigen-binding fragment thereof that comprises an amino acid sequence of SEQ ID NO: 23 or an amino acid sequence having 85%, 90% or 95% sequence identity thereto, wherein the binder comprises a CDR1 of SEQ ID NO: 3, a CDR2 of SEQ ID NO: 8, and a CDR3 of SEQ ID NO: 12.

In a further particular embodiment, the binder of the present invention comprises an antibody or antigen-binding fragment thereof that comprises an amino acid sequence of SEQ ID NO: 24 or an amino acid sequence having 85%, 90% or 95% sequence identity thereto, wherein the binder comprises a CDR1 of SEQ ID NO: 4, a CDR2 of SEQ ID NO: 8, and a CDR3 of SEQ ID NO: 12.

In a yet further particular embodiment, the binder of the present invention comprises an antibody or antigen-binding fragment thereof that comprises an amino acid sequence of SEQ ID NO: 25 or an amino acid sequence having 85%, 90% or 95% sequence identity thereto, wherein the binder comprises a CDR1 of SEQ ID NO: 5, a CDR2 of SEQ ID NO: 9, and a CDR3 of SEQ ID NO: 13.

In another aspect, the invention provides a binder, such as an antibody or antigen-binding fragment thereof, that competes for specific binding to hCCR8 with a binder as described herein. In particular with a hCCR8 single-domain antibody moiety having the amino acid sequence of SEQ ID NO: 35, 36, 37, or 38. Therefore, in a particular embodiment, the present invention provides a blocking hCCR8 binder that competes for specific binding to hCCR8 with a single-domain antibody having the amino acid sequence of SEQ ID NO: 35, 36, 37, or 38. In another particular embodiment with a hCCR8 single-domain antibody moiety having the amino acid sequence of SEQ ID NO: 21, 22, 23, 24, or 25. Therefore, in a particular embodiment, the present invention provides a blocking hCCR8 binder that competes for specific binding to hCCR8 with a single-domain antibody having the amino acid sequence of SEQ ID NO: 21, 22, 23, 24, or 25. It can easily be determined if a binder competes for specific binding to hCCR8 with a binder as described herein using routine methods known in the art. For example, to determine whether a test binder competes, the binder of the invention is allowed to bind to a hCCR8 protein under saturation conditions.

Next, the ability of the test binder is evaluated. If the test binder cannot bind to the hCCR8 protein, it can be concluded that the test antibody competes with the binder of the invention for specific binding to hCCR8.

Cytotoxicity

Another aspect of the present invention is to provide a human CCR8 binder having cytotoxic activity. “Cytotoxicity” or “cytotoxic activity” as used herein refers to the ability of a binder to be toxic to a cell that it is bound to. As is clear to the skilled person from the description of the invention, any type of cytotoxicity can be used in the context of the invention. Cytotoxicity can be direct cytotoxicity, wherein the binder itself directly damages the cell (e.g. because it comprises a chemotherapeutic payload) or it can be indirect, wherein the binder induces extracellular mechanisms that cause damage to the cell (e.g. an antibody that induces antibody-dependent cellular activity). More in particular, the binder of the invention can signal the immune system to destroy or eliminate the cell it is bound to or the binder can carry a cytotoxic payload to destroy the cell it is bound to. In particular, the cytotoxic activity is caused by the presence of cytotoxic moiety. Examples of such cytotoxic moieties includes moieties which induce antibody-dependent cellular activity (ADCC), induce antibody-dependent cellular phagocytosis (ADCP), induce complement-dependent cytotoxicity (CDC), bind to and activate T-cells, or comprise a cytotoxic payload. Most preferably, said cytotoxic moiety induces antibody-dependent cellular activity (ADCC).

Antibody-dependent cellular cytotoxicity (ADCC) refers to a cell-mediated reaction in which non-specific cytotoxic cells that express Fc receptors recognize binders on a target cell and subsequently cause lysis of the target cell. Examples of non-specific cytotoxic cells that express Fc receptors include natural killer cells, neutrophils, monocytes and macrophages.

Complement-dependent cytotoxicity (CDC) refers to the lysis of a target in the presence of complement. The complement activation pathway is initiated by the binding of the first component of the complement system (C1q) to a binder complexed with a cognate antigen.

Antibody-dependent cellular phagocytosis (ADCP) refers to a cell-mediated reaction in which phagocytes (such as macrophages) that express Fc receptors recognize binders on a target cell and thereby lead to phagocytosis of the target cell.

CDC, ADCC and ADCP can be measured using assays that are known in the art (Vafa et al. Methods 2014 Jan. 1; 65(1):114-26 (2013)).

Binding to and activation of T-cells refers to the binding of a T-cell marker that is distinct from hCCR8 and the resulting activation of said T-cell. Activation of the T-cell induces the cytotoxic activity of the T-cell against the cell on which the binder of the invention is bound. Therefore, in a particular embodiment, the binder of the invention binds to hCCR8 and binds to and activates T-cells. For example, the cytotoxic moiety may bind to hCD3. In a further embodiment, the cytotoxic moiety comprises an antibody or antigen-binding fragment thereof that binds to hCD3. Thus, the binder of the invention may bind to hCCR8 and hCD3. Such a binder binds to intratumoural Tregs and directs the cytotoxic activity of T-cells to these Tregs, thereby depleting them from the tumour environment. In a particular embodiment, the binder of the invention comprises a moiety that binds to hCCR8 and a moiety that binds to hCD3, wherein at least one moiety is antibody based, particularly wherein both moieties are antibody based. Therefore, in a particular embodiment, the present invention provides a bispecific construct comprising an antibody or antigen-binding fragment thereof that specifically binds to hCCR8 and an antibody or antigen-binding fragment thereof that specifically binds to hCD3.

A cytotoxic payload refers to any molecular entity that causes a direct damaging effect on the cell that is contacted with the cytotoxic payload. Cytotoxic payloads are known to the persons skilled in the art. In a particular embodiment, the cytotoxic payload is a chemical entity. Particular examples of such cytotoxic payloads include toxins, chemotherapeutic agents and radioisotopes or radionuclides. In a further embodiment, the cytotoxic payload comprises an agent selected from the group consisting of alkylating agents, anthracyclines, cytoskeletal disruptors, epothilones, histone deacetylase inhibitors, inhibitors of topoisomerase I, inhibitors of topoisomerase II, kinase inhibitors, nucleotide analogues and precursor analogues, peptide antibiotics, platinum-based agents, retinoids, vinca alkaloids and derivatives, peptide or small molecule toxins, and radioisotopes. Chemical entities can be coupled to proteinaceous inhibitors, e.g. antibodies or antigen-binding fragments, using techniques known in the art. Such coupling can be covalent or non-covalent and the coupling can be labile or reversible.

As is well known in the field, the Fc region of IgG antibodies interacts with several cellular Fcγ receptors (FcγR) to stimulate and regulate downstream effector mechanisms. There are five activating receptors, namely FcγRI (CD64), FcγRIIa (CD32a), FcγRIIc (CD32c), FcγRIIIa (CD16a) and FcγRIIIb (CD16b), and one inhibitory receptor FcγRIIb (CD32b). The communication of IgG antibodies with the immune system is controlled and mediated by FcγRs, which relay the information sensed and gathered by antibodies to the immune system, providing a link between the innate and adaptive immune systems, and particularly in the context of biotherapeutics (Hayes J et al., 2016. J Inflamm Res 9: 209-219).

IgG subclasses vary in their ability to bind to FcγR and this differential binding determines their ability to elicit a range of functional responses. For example, in humans, FcγRIIIa is the major receptor involved in the activation of antibody-dependent cell-mediated cytotoxicity (ADCC) and IgG3 followed closely by IgG1 display the highest affinities for this receptor, reflecting their ability to potently induce ADCC. Whilst IgG2 have been shown to have weaker binding for this receptor binders having the human IgG2 isotype have also been found to efficiently deplete Tregs.

In a preferred embodiment of the invention, the binder of the invention induces antibody effector function, in particular antibody effector function in human. In a particular embodiment, the binder of the invention binds FcγR with high affinity, preferably an activating receptor with high affinity. Preferably the binder binds FcγRI and/or FcγRIIa and/or FcγRIIIa with high affinity. Particularly preferably, the binder binds to FcγRIIIa. In a particular embodiment, the binder binds to at least one activating Fcγ receptor with a dissociation constant of less than about 10⁻⁶ M, 10⁻⁷ M, 10⁻⁸ M, 10⁻⁹ M, 10⁻¹° M, 10⁻¹¹ M, 10⁻¹² M or 10⁻¹³ M. FcγR binding can be obtained through several means. For example, the cytotoxic moiety may comprise a fragment crystallisable (Fc) region moiety or it may comprise a binding part, such as an antibody or antigen-binding part thereof that specifically binds to an FcγR.

In one particular embodiment, the cytotoxic moiety comprises a fragment crystallisable (Fc) region moiety. Preferably, the Fc region moiety is an IgG Fc domain derived from IgG1, IgG2, IgG3 and IgG4 antibody. More preferably, the Fc region moiety is an IgG Fc domain derived from a human IgG1 antibody. More preferably, the Fc region moiety is an IgG Fc domain derived from a short hinge variant of a human IgG1 antibody. In a particular embodiment, the Fc region moiety comprises the amino acid sequence of SEQ ID NO: 78 or an amino acid sequence having at least 80% sequence identity, such as at least 85% sequence identity or 90% sequence identity. In a further embodiment, an amino acid sequence of SEQ ID NO: 78 or an amino acid sequence having at least 95%, 96%, 97% sequence identity, in particular at least 98% sequence identity, more in particular at least 99% sequence identity.

In one embodiment, the Fc region moiety has been engineered to increase ADCC, ADCP and/or CDC activity.

ADCC may be increased by methods that reduce or eliminate the fucose moiety from the Fc moiety glycan and/or through introduction of specific mutations on the Fc region of an immunoglobulin, such as IgG1 (e.g. S298A/E333/K334A, S239D/1332E/A330L or G236A/S239D/A330U1332E) (Lazar et al. Proc Natl Acad Sci USA 103:2005-2010 (2006); Smith et al. Proc Natl Acad Sci USA 209:6181-6 (2012)). ADCP may also be increased by the introduction of specific mutations on the Fc portion of human IgG (Richards et al. Mol Cancer Ther 7:2517-27 (2008)). Methods for engineering binders for increased ADCC, CDC and ADCP activity have been described in Saunders (Frontiers in Immunology 2019, 1296) and Wang et al. (Protein Cell 2019, 9:63-73).

In a particular embodiment of the present invention, the binder comprising an Fc region moiety is optimized to elicit an ADCC response, that is to say the ADCC response is enhanced, increased or improved relative to other hCCR8 binders comprising an Fc region moiety, including those that are cross-reactive with murine CCR8 and, for example, unmodified anti-CCR8 monoclonal antibodies. In a preferred embodiment, the hCCR8 binder has been engineered to elicit an enhanced ADCC response.

In a preferred embodiment of the present invention, the binder comprising an Fc region moiety is optimized to elicit an ADCP response, that is to say the ADCP response is enhanced, increased or improved relative to other hCCR8 binders comprising an Fc region moiety, including those that are cross-reactive with murine CCR8 and, for example, unmodified anti-hCCR8 monoclonal antibodies.

In another embodiment, the cytotoxic moiety comprises a moiety that binds to an Fc gamma receptor. More in particular binds to and activates an FcγR, in particular an activating receptor, such as FcγRI and/or FcγRIIa and/or FcγRIIIa, especially FcγRIIIa. The moiety that binds to an FcγR may be antibody based or non-antibody based as described herein before. If antibody based, the moiety may bind the FcγR through its variable region.

In a further embodiment of the present invention, the hCCR8 binder, as detailed above comprises at least one Fc region moiety and a single-domain antibody moiety that binds to hCCR8, as detailed above.

In one embodiment of the present invention, the hCCR8 binder is a genetically engineered polypeptide that comprises at least one Fc region moiety and a single-domain antibody moiety that binds to hCCR8, joined together by a direct bond.

In another embodiment, the hCCR8 binder is a genetically engineered polypeptide that comprises at least one Fc region moiety and a single-domain antibody moiety that binds to hCCR8, joined together by a direct bond or a linker. Preferably, the linker is a peptide linker. Preferably, the peptide linker is a flexible linker having an amino acid sequence consisting primarily of stretches of Glycine (G) and Serine (S) residues (a so-called “GS” or “GlySer” linker). In a further embodiment, at least 80%, in particular at least 85%, more in particular at least 90% of amino acid residues in the peptide linker are selected from glycine and serine. In a preferred embodiment comprising at least 95% of amino acid residues in the peptide linker are selected from glycine and serine. In another particular embodiment, the peptide linker comprises from 1 to 50 amino acids, such as from 1 to 40, in particular from 1 to 30. In a particular embodiment from 5 to 25 amino acids, preferably from 8 to 22 amino acids, such as from 10 to 20 amino acids. A preferred example of such a GS linker comprises the sequence of GGGGS (SEQ ID NO: 50). In such a linker, the sequence of SEQ ID NO: 50 can be repeated “n” times to optimize the length of the GS linker to achieve appropriate properties of the binder, so that the sequence of the linker will be that of (SEQ ID NO: 50)ₙ. Typically the copy number “n” ranges from 1 to 10, or from 2 to 4. The amino acid sequence of the Fc region moiety and/or the single domain antibody moiety region(s) may be humanized to reduce immunogenicity for humans.

Therefore, in a particular embodiment, the hCCR8 binder of the invention has the formula B-L-C; wherein B refers to a hCCR8 binding moiety as described herein, L refers to a linker as described herein, and C refers to a cytotoxic moiety as described herein. As will be understood from the disclosures herein, preferably B comprises a single-domain antibody moiety that binds to hCCR8, L is either a direct bond or has the sequence (SEQ ID NO: 50)ₙ wherein n is an integer from 1 to 10, and C is an Fc region moiety.

In one embodiment, the hCCR8 binder of the invention has the formula B-L-C, wherein B is a single-domain antibody moiety corresponding to any of SEQ ID NO: 21 to 25 or 35 to 38, L is a linker corresponding to (SEQ ID NO: 50)ₙ, wherein “n” ranges from 1 to 10, and C is an Fc region moiety.

Preferably, the hCCR8 binder of the invention has the formula B-L-C, wherein B is a single-domain antibody moiety corresponding to any of SEQ ID NO: 21 to 25 or 35 to 38, L is a linker corresponding to (SEQ ID NO: 50)ₙ, wherein “n” is 2 or 4, and C is an IgG Fc domain derived from a short hinge variant of a human IgG1 antibody.

More preferably, said hCCR8 binder, as detailed above comprises the amino acid sequences corresponding to any of SEQ ID NO: 60 to 77.

In a further embodiment, the hCCR8 binder of the invention has the formula B-C, wherein B is a single-domain antibody moiety corresponding to any of SEQ ID NO: 21 to 25 or 35 to 38, and C is an Fc region moiety.

Preferably, the hCCR8 binder of the invention has the formula B-C, wherein B is a single-domain antibody moiety corresponding to SEQ ID NO: 21 to 25 or 35 to 38 and C is an IgG Fc domain derived from a short hinge variant of a human IgG1 antibody.

More preferably, said hCCR8 binder, as detailed above comprises the amino acid sequences corresponding to any one of SEQ ID NO: 51 to 59.

As described herein before, in a particular embodiment, C comprises the sequence of SEQ ID NO: 78.

In a further embodiment, the present invention provides nucleic acid molecules encoding hCCR8 binders as defined herein. In some embodiments, such provided nucleic acid molecules may contain codon-optimized nucleic acid sequences. In another embodiment, the nucleic acid is included in an expression cassette within appropriate nucleic acid vectors for the expression in a host cell such as, for example, bacterial, yeast, insect, piscine, murine, simian, or human cells. In some embodiments, the present invention provides host cells comprising heterologous nucleic acid molecules (e.g. DNA vectors) that express the desired binder.

In a particular embodiment, the binder of the invention is administered as a therapeutic nucleic acid. The term “therapeutic nucleic acid” used herein refers to any nucleic acid molecule that have a therapeutic effect when introduced into a eukaryotic organism (e.g., a mammal such as human) and includes DNA and RNA molecules encoding the binder of the invention. As is known to the skilled person, the nucleic acid may comprise elements that induce transcription and/or translation of the nucleic acid or that increases ex and/or in vivo stability of the nucleic acid.

In some embodiments, the present invention provides methods of preparing an isolated hCCR8 binder as defined above. In some embodiments, such methods may comprise culturing a host cell that comprises nucleic acids (e.g. heterologous nucleic acids that may comprise and/or be delivered to the host cell via vectors). Preferably, the host cell (and/or the heterologous nucleic acid sequences) is/are arranged and constructed so that the binder is secreted from the host cell and isolated from cell culture supernatants.

Treatment

A hCCR8 binder presenting the features as described herein represents a further object of the invention. The hCCR8 binder can be used as a medicine. In a further embodiment the invention provides a method for treating a disease in a subject comprising administering an hCCR8 binder having cytotoxic activity. Preferably the disease is a cancer, in particular a solid tumour.

In a preferred embodiment of the present invention, the subject of the aspects of the invention as described herein, is a mammal, preferably a cat, dog, horse, donkey, sheep, pig, goat, cow, hamster, mouse, rat, rabbit, or guinea pig, but most preferably the subject is a human. Thus in all aspects of the invention as described herein the subject is preferably a human.

As used herein, the terms “cancer”, “cancerous”, or “malignant” refer to or describe the physiological condition on mammals that is typically characterized by unregulated cell growth.

As used herein, the term “tumour” as it applies to a subject diagnosed with, or suspected of having, a cancer refers to a malignant or potentially malignant neoplasm or tissue mass of any size, and includes primary tumours and secondary neoplasms. The terms “cancer”, “malignancy”, “neoplasm”, “tumour” and “carcinoma” can also be used interchangeably herein to refer to tumours and tumour cells that exhibit an aberrant growth phenotype characterized by a significant loss of control of cell proliferation. In general, cells of interest for treatment include precancerous (e.g. benign), malignant, pre-metastatic, metastatic, and non-metastatic cells. The teachings of the present disclosure may be relevant to any and all tumours.

Examples of tumours include but are not limited to, carcinoma, lymphoma, leukemia, blastoma, and sarcoma. More particular examples of such cancers include squamous cell carcinoma, myeloma, small-cell lung cancer, non-small cell lung cancer, glioma, hepatocellular carcinoma (HCC), hodgkin's lymphoma, non-hodgkin's lymphoma, acute myeloid leukemia (AML), anaplastic large cell lymphoma (ALCL), cutaneous T-cell lymphoma (CTCL), Adult T-cell leukemia/lymphoma (ATLL), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma and head and neck cancer.

In one aspect, the tumour involves a solid tumour. Examples of solid tumours are sarcomas (including cancers arising from transformed cells of mesenchymal origin in tissues such as cancellous bone, cartilage, fat, muscle, vascular, hematopoietic, or fibrous connective tissues), carcinomas (including tumours arising from epithelial cells), mesothelioma, neuroblastoma, retinoblastoma, etc. Tumours involving solid tumours include, without limitations, brain cancer, lung cancer, stomach cancer, duodenal cancer, esophagus cancer, breast cancer, colon and rectal cancer, renal cancer, bladder cancer, kidney cancer, pancreatic cancer, prostate cancer, ovarian cancer, melanoma, mouth cancer, sarcoma, eye cancer, thyroid cancer, urethral cancer, vaginal cancer, neck cancer, lymphoma, and the like.

In another particular embodiment, the tumour is selected from the group consisting of breast invasive carcinoma, colon adenocarcinoma, head and neck squamous carcinoma, stomach adenocarcinoma, lung adenocarcinoma (NSCLC), lung squamous cell carcinoma (NSCLC), kidney renal clear cell carcinoma, skin cutaneous melanoma, esophageal cancer, cervical cancer, hepatocellular carcinoma, merkel cell carcinoma, small Cell Lung Cancer (SCLC), classical Hodgkin Lymphoma (cHL), urothelial Carcinoma, Microsatellite Instability-High (MSI-H) Cancer and mismatch repair deficient (dMMR) cancer.

In a further embodiment, the tumour is selected from the group consisting of a breast cancer, uterine corpus cancer, lung cancer, stomach cancer, head and neck squamous cell carcinoma, skin cancer, colorectal cancer, and kidney cancer. In an even further embodiment, the tumour is selected from the group consisting of breast invasive carcinoma, colon adenocarcinoma, head and neck squamous carcinoma, stomach adenocarcinoma, lung adenocarcinoma (NSCLC), lung squamous cell carcinoma (NSCLC), kidney renal clear cell carcinoma, and skin cutaneous melanoma. In one aspect, the cancers involve CCR8 expressing tumours, including but not limited to breast cancer, uterine corpus cancer, lung cancer, stomach cancer, head and neck squamous cell carcinoma, skin cancer, colorectal cancer, and kidney cancer. In one particular embodiment, the tumour is selected from the group consisting of breast cancer, colon adenocarcinoma, and lung carcinoma.

In a particular embodiment, the tumour is a T-cell lymphoma, in particular a T-cell lymphoma expressing CCR8 including, but not limited to Adult T-cell leukemia/lymphoma (ATLL), cutaneous T-cell lymphoma (CTCL) and anaplastic large cell lymphoma (ALCL).

In a further embodiment, the tumour is a tumour carrying recurrent chromosomal rearrangements involving the DUSP22-IRF4 locus on 6p25.3 (so-called DUSP22 rearrangements). Preferably, the tumour is a lymphoma carrying DUSP22 rearrangements.

As used herein, the term “administration” refers to the act of giving a drug, prodrug, antibody, or other agent, or therapeutic treatment to a physiological system (e.g. a subject or in vivo, in vitro, or ex vivo cells, tissues, and organs). Exemplary routes of administration to the human body can be through the mouth (oral), skin (transdermal), oral mucosa (buccal), ear, by injection (e.g. intravenously, subcutaneously, intratumourally, intraperitoneally, etc.) and the like. The term administration of the binder of the invention includes direct administration of the binder as well as indirect administration by administering a nucleic acid encoding the binder such that the binder is produced from the nucleic acid in the subject. Administration of the binder thus includes DNA and RNA therapy methods that result in in vivo production of the binder.

Reference to “treat” or “treating” a tumour as used herein defines the achievement of at least one therapeutic effect, such as for example, reduced number of tumour cells, reduced tumour size, reduced rate to cancer cell infiltration into peripheral organs, or reduced rate of tumour metastasis or tumour growth. As used herein, the term “modulate” refers to the activity of a compound to affect (e.g. to promote or treated) an aspect of the cellular function including, but not limited to, cell growth, proliferation, invasion, angiogenesis, apoptosis, and the like.

Positive therapeutic effects in cancer can be measured in a number of ways (e.g. Weber (2009) J Nucl Med 50, 1S-10S). By way of example, with respect to tumour growth inhibition, according to National Cancer Institute (NCI) standards, a T/C42% is the minimum level of anti-tumour activity. A T/C<10% is considered a high anti-tumour activity level, with T/C (%)=Median tumour volume of the treated/Median tumour volume of the control×100. In some embodiments, the treatment achieved by a therapeutically effective amount is any of progression free survival (PFS), disease free survival (DFS) or overall survival (OS). PFS, also referred to as “Time to Tumour Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients.

Reference to “prevention” (or prophylaxis) as used herein refers to delaying or preventing the onset of the symptoms of the cancer. Prevention may be absolute (such that no disease occurs) or may be effective only in some individuals or for a limited amount of time.

In a preferred aspect of the invention the subject has an established tumour, that is the subject already has a tumour, e.g. that is classified as a solid tumour. As such, the invention as described herein can be used when the subject already has a tumour, such as a solid tumour. As such, the invention provides a therapeutic option that can be used to treat an existing tumour. In one aspect of the invention the subject has an existing solid tumour. The invention may be used as a prevention, or preferably as a treatment in subjects who already have a solid tumour. In one aspect the invention is not used as a preventative or prophylaxis.

In one aspect, tumour regression may be enhanced, tumour growth may be impaired or reduced, and/or survival time may be enhanced using the invention as described herein, for example compared with other cancer treatments (for example standard-of care treatments for the a given cancer).

In one aspect of the invention the method of treatment or prevention of a tumour as described herein further comprises the step of identifying a subject who has tumour, preferably identifying a subject who has a solid tumour.

The dosage regimen of a therapy described herein that is effective to treat a patient having a tumour may vary according to factors such as the disease state, age, and weight of the patient, and the ability of the therapy to elicit an anti-cancer response in the subject. Selection of an appropriate dosage will be within the capability of one skilled in the art. For example 0.01, 0.1, 0.3, 0.5, 1, 2, 3, 4, 5, 6, 7, 8, 9, 10, 20, 30, 40, or 50 mg/kg. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen).

The binder according to any aspect of the invention or the nucleic acid encoding it as described herein may be in the form of a pharmaceutical composition which additionally comprises a pharmaceutically acceptable carrier, diluent or excipient. As used herein, the term “pharmaceutically acceptable carrier” or “pharmaceutically acceptable excipient” includes any material which, when combined with an active ingredient, allows the ingredient to retain biological activity. Pharmaceutically acceptable carriers enhance or stabilize the composition or can be used to facilitate preparation of the composition. Pharmaceutically acceptable carriers include solvents, dispersion media, coatings, antibacterial and antifungal agents, isotonic and absorption delaying agents, and the like that are physiologically compatible, as is known to those skilled in the art (see, for example, Remington's Pharmaceutical Sciences, 18th Ed. Mack Printing Company, 1990, pp. 1289-1329; Remington: The Science and Practice of Pharmacy, 21st Ed. Pharmaceutical Press 2011; and subsequent versions thereof). Non-limiting examples of said pharmaceutically acceptable carrier comprise any of the standard pharmaceutical carriers such as a phosphate buffered saline solution, water, emulsions such as oil/water emulsion, and various types of wetting agents. Therefore, the present invention further provides the use of a binder of the invention in the manufacture of a medicament for the treatment of a tumour.

These compositions include, for example, liquid, semi-solid and solid dosage formulations, such as liquid solutions (e.g., injectable and infusible solutions), dispersions or suspensions, tablets, pills, or liposomes. In some embodiments, a preferred form may depend on the intended mode of administration and/or therapeutic application. Pharmaceutical compositions containing the binder or the nucleic acid of the invention can be administered by any appropriate method known in the art, including, without limitation, oral, mucosal, by-inhalation, topical, buccal, nasal, rectal, or parenteral (e.g. intravenous, infusion, intratumoural, intranodal, subcutaneous, intraperitoneal, intramuscular, intradermal, transdermal, or other kinds of administration involving physical breaching of a tissue of a subject and administration of the pharmaceutical composition through the breach in the tissue). Such a formulation may, for example, be in a form of an injectable or infusible solution that is suitable for intradermal, intratumoural or subcutaneous administration, or for intravenous infusion. In a particular embodiment, the binder or nucleic acid is administered intravenously. The administration may involve intermittent dosing. Alternatively, administration may involve continuous dosing (e.g., perfusion) for at least a selected period of time, simultaneously or between the administration of other compounds.

Formulations of the invention generally comprise therapeutically effective amounts of a binder of the invention. “Therapeutic levels”, “therapeutically effective amount” or “therapeutic amount” means an amount or a concentration of an active agent that has been administered that is appropriate to safely treat the condition to reduce or prevent a symptom of the condition.

In some embodiments, the binder can be prepared with carriers that protect it against rapid release and/or degradation, such as a controlled release formulation, such as implants, transdermal patches, and microencapsulated delivery systems. Biodegradable, biocompatible polymers can be used.

Those skilled in the art will appreciate, for example, that route of delivery (e.g., oral vs intravenous vs subcutaneous vs intratumoural, etc) may impact dose amount and/or required dose amount may impact route of delivery. For example, where particularly high concentrations of an agent within a particular site or location (e.g., within a tumour) are of interest, focused delivery (e.g., in this example, intratumoural delivery) may be desired and/or useful. Other factors to be considered when optimizing routes and/or dosing schedule for a given therapeutic regimen may include, for example, the particular cancer being treated (e.g., type, stage, location, etc.), the clinical condition of a subject (e.g., age, overall health, etc.), the presence or absence of combination therapy, and other factors known to medical practitioners.

The pharmaceutical compositions typically should be sterile and stable under the conditions of manufacture and storage. The composition can be formulated as a solution, microemulsion, dispersion, liposome, or other ordered structure suitable to high drug concentration. Sterile injectable solutions can be prepared by incorporating the binder in the required amount in an appropriate solvent with one or a combination of ingredients enumerated above, as required, followed by filtered sterilization. Formulations for parenteral administration include, but are not limited to, suspensions, solutions, emulsions in oily or aqueous vehicles, pastes, and implantable sustained-release or biodegradable formulations as discussed herein. Sterile injectable formulations may be prepared using a non-toxic parenterally acceptable diluent or solvent. Each pharmaceutical composition for use in accordance with the present invention may include pharmaceutically acceptable dispersing agents, wetting agents, suspending agents, isotonic agents, coatings, antibacterial and antifungal agents, carriers, excipients, salts, or stabilizers are non-toxic to the subjects at the dosages and concentrations employed. Preferably, such a composition can further comprise a pharmaceutically acceptable carrier or excipient for use in the treatment of cancer that that is compatible with a given method and/or site of administration, for instance for parenteral (e.g. sub-cutaneous, intradermal, or intravenous injection), intratumoural, or peritumoural administration.

While an embodiment of the treatment method or compositions for use according to the present invention may not be effective in achieving a positive therapeutic effect in every subject, it should do so in a using pharmaceutical compositions and dosing regimens that are consistently with good medical practice and statistically significant number of subjects as determined by any statistical test known in the art such as the Student's t-test, the X²-test, the U-test according to Mann and Whitney, the Kruskal-Wallis test (H-test), Jonckheere-Terpstra test and the Wilcoxon-test.

Where hereinbefore and subsequently a tumour, a tumour disease, a carcinoma or a cancer is mentioned, also metastasis in the original organ or tissue and/or in any other location are implied alternatively or in addition, whatever the location of the tumour and/or metastasis is.

As discussed herein, the present invention relates to depleting regulatory T cells (Tregs). Thus, in one aspect of the invention, treatment with the hCCR8 binder having cytotoxic activity depletes or reduces regulatory T cells, especially tumour-infiltrating regulatory T cells. In one aspect, the depletion is via ADCC. In another aspect, the depletion is via CDC. In a further aspect, the depletion is via ADCP.

As such, the invention provides a method for depleting regulatory T cells in a tumour in a subject, comprising administering to said subject a hCCR8 binder having cytotoxic activity. In a preferred embodiment Tregs are depleted in a solid tumour. By “depleted” it is meant that the number, ratio or percentage of Tregs is decreased relative to when the hCCR8 binder having cytotoxic activity, is not administered. In particular embodiments of the invention as described herein, over about 5%, 10%, 20%, 30%, 40%, 50%, 60%, 70%, 80%, 90% or 99% of the tumour-infiltrating regulatory T cells are depleted.

As used herein, “regulatory T cells” (“Treg”, “Treg cells”, or “Tregs”) refer to a lineage of CD4+T lymphocytes specialized in controlling autoimmunity, allergy and infection. Typically, they regulate the activities of T cell populations, but they can also influence certain innate immune system cell types. Tregs are usually identified by the expression of the biomarkers CD3, CD4, CD25, and CD127 or Foxp3. Naturally occurring Treg cells normally constitute about 5-10% of the peripheral CD4+T lymphocytes. However, within a tumour microenvironment (i.e. tumour-infiltrating Treg cells), they can make up as much as 20-30% of the total CD4+T lymphocyte population.

Activated human Treg cells may directly kill target cells such as effector T cells and APCs through perforin- or granzyme B-dependent pathways; cytotoxic T-lymphocyte-associated antigen 4 (CTLA4+) Treg cells induce indoleamine 2,3-dioxygenase (IDO) expression by APCs, and these in turn suppress T-cell activation by reducing tryptophan; Treg cells, may release interleukin-10 (IL-10) and transforming growth factor (TGFβ) in vivo, and thus directly inhibit T-cell activation and suppress APC function by inhibiting expression of MHC molecules, CD80, CD86 and IL-12. Treg cells can also suppress immunity by expressing high levels of CTLA4 which can bind to CD80 and CD86 on antigen presenting cells and prevent proper activation of effector T cells. It is furthermore known that Treg cells express high levels of CD25, thereby competing with IL2 binding to CD8 and reducing CD8-induced proliferation and survival.

In a preferred embodiment of the present invention the ratio of effector T cells to regulatory T cells in a solid tumour is increased after administration of the binder of the invention. In some embodiments, the ratio of effector T cells to regulatory T cells in a solid tumour is increased to over 5, 10, 15, 20, 40 or 80.

An immune effector cell refers to an immune cell which is involved in the effector phase of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils.

Immune effector cells involved in the effector phase of an immune response express specific Fc receptors and carry out specific immune functions. An effector cell can induce antibody-dependent cell-mediated cytotoxicity (ADCC), e.g., a neutrophil capable of inducing ADCC. For example, monocytes, macrophages, neutrophils, eosinophils, and lymphocytes which express FcaR are involved in specific killing of target cells and presenting antigens to other components of the immune system, or binding to cells that present antigens. An effector cell can also phagocytose a target antigen, target cell, or microorganism. As discussed herein, antibodies according to the present invention may be optimised for ability to induce ADCC.

In preferred embodiments, the methods and compositions for depleting Tregs are specific for Tregs with limited to no impact on other T cells. In further embodiments, the methods and compositions of the present invention deplete tumour-infiltrating Tregs to a greater extent than other Tregs. In a further embodiment, the methods and compositions of the present invention deplete tumour-infiltrating Tregs to a greater extent than circulating Tregs. In yet another embodiment, the methods and compositions of the present invention deplete tumour-infiltrating Tregs to a greater extent than normal tissue-infiltrating Tregs, such as intestinal Tregs. Comparing the extent of depletion of cell populations is preferably performed by comparing the percentage decrease of the cell population without and with treatment, such as shown in the examples.

In a further particular embodiment, the methods and compositions of the invention decrease the ratio of Tregs over T-cells, in particular the ratio of Tregs over T-cells in a tumour. In a further embodiment, the methods and compositions of the invention decrease the ratio of Tregs over T-cells in the tumour to a greater extent than the ratio of Tregs over T-cells outside of the tumour. In another embodiment, the methods and compositions of the invention decrease the ratio of Tregs over T-cells in the tumour to a greater extent than the ratio of Tregs over T-cells in normal tissue, in particular in intestinal tissue.

In yet a further aspect of the present invention, treatment with the hCCR8 binder having cytotoxic activity or the nucleic acid encoding the same as described herein depletes or reduces any type of cells expressing hCCR8. Preferably, the cells are tumour cells expressing hCCR8. Therefore, the invention provides a method for depleting cells, preferably tumour cells, in a subject, comprising administering to said subject a hCCR8 binder having cytotoxic activity or the nucleic acid encoding the same as described herein.

In some embodiments, a different agent against cancer may be administered in combination with the binder of the invention via the same or different routes of delivery and/or according to different schedules. Alternatively or additionally, in some embodiments, one or more doses of a first active agent is administered substantially simultaneously with, and in some embodiments via a common route and/or as part of a single composition with, one or more other active agents. Those skilled in the art will further appreciate that some embodiments of combination therapies provided in accordance with the present invention achieve synergistic effects; in some such embodiments, dose of one or more agents utilized in the combination may be materially different (e.g., lower) and/or may be delivered by an alternative route, than is standard, preferred, or necessary when that agent is utilized in a different therapeutic regimen (e.g., as monotherapy and/or as part of a different combination therapy).

In some embodiments, where two or more active agents are utilized in accordance with the present invention, such agents can be administered simultaneously or sequentially. In some embodiments, administration of one agent is specifically timed relative to administration of another agent. For example, in some embodiments, a first agent is administered so that a particular effect is observed (or expected to be observed, for example based on population studies showing a correlation between a given dosing regimen and the particular effect of interest). In some embodiments, desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular patient of interest.

In another aspect of the invention, an hCCR8 binder has improved therapeutic effects when combined with an immune checkpoint inhibitor. A combination therapy with a hCCR8 binder and an immune checkpoint inhibitor can have synergistic effects in the treatment of established tumours. As such, the interaction between the PD-1 receptor and the PD-L1 ligand may be blocked, resulting in “PD-1 blockade”. In one aspect, the combination may lead to enhanced tumour regression, enhanced impairment or reduction of tumour growth, and/or survival time may be enhanced using the invention as described herein, for example compared with administration of the checkpoint inhibitor alone. Therefore, in a particular aspect of the invention, the present invention provides a hCCR8 binder of the invention for use in the treatment of a tumour, wherein the treatment further comprises administration of an immune checkpoint inhibitor.

As used herein, “immune checkpoint” or “immune checkpoint protein” refer to proteins belonging to inhibitory pathways in the immune system, in particular for the modulation of T-cell responses. Under normal physiological conditions, immune checkpoints are crucial to preventing autoimmunity, especially during a response to a pathogen. Cancer cells can alter the regulation of the expression of immune checkpoint proteins in order to avoid immune surveillance.

Examples of immune checkpoint proteins include but are not limited to PD-1, CTLA-4, BTLA, KIR, CD155, B7H4, VISTA and TIM3, and also OX40, GITR, 4-1BB and HVEM. Immune checkpoint proteins may also refer to proteins which bind to other immune checkpoint proteins. Such proteins include PD-L1, PD-L2, CD80, CD86, HVEM, LLT1, and GAL9.

“Immune checkpoint protein inhibitor”, “immune checkpoint inhibitor”, or “checkpoint inhibitor” refers to any molecule that can interfere with the signalling and/or protein-protein interactions mediated by an immune checkpoint protein. In one aspect of the invention the immune checkpoint protein is PD-1 or PD-L1. In a preferred aspect of the invention as described herein the immune checkpoint inhibitor interferes with PD-1/PD-L1 interactions via anti-PD-1 or anti PD-L1 antibodies.

In another particular embodiment, the immune checkpoint is CTLA-4 (also known as CTLA4, cytotoxic T-lymphocyte-associated protein 4 or CD152) and the immune checkpoint inhibitor is an inhibitor of CTLA-4. In a particular embodiment, the binder of the invention is used in the treatment of a tumour, wherein the treatment further comprises administration of a CTLA-4 inhibitor, in particular an anti-CTLA-4 antibody, particularly a blocking anti-CTLA-4 antibody. Anti-CTLA-4 antibodies of the instant invention can bind to an epitope on human CTLA-4 so as to inhibit CTLA-4 from interacting with a human B7 counter-receptor. Because interaction of human CTLA-4 with human B7 transduces a signal leading to inactivation of T-cells bearing the human CTLA-4 receptor, antagonism of the interaction effectively induces, augments or prolongs the activation of T cells bearing the human CTLA-4 receptor, thereby prolonging or augmenting an immune response. Anti-CTLA-4 antibodies are described in U.S. Pat. Nos. 5,811,097; 5,855,887; 6,051,227; in PCT Application Publication Nos. WO 01/14424 and WO 00/37504; and in U.S. Patent Publication No. 2002/0039581. Each of these references is specifically incorporated herein by reference for purposes of description of anti-CTLA-4 antibodies. An exemplary clinical anti-CTLA-4 antibody is human monoclonal antibody 10D1 as disclosed in WO 01/14424 and U.S. patent application Ser. No. 09/644,668. Antibody 10D1 has been administered in single and multiple doses, alone or in combination with a vaccine, chemotherapy, or interleukin-2 to more than 500 patients diagnosed with metastatic melanoma, prostate cancer, lymphoma, renal cell cancer, breast cancer, ovarian cancer, and HIV. Other anti-CTLA-4 antibodies encompassed by the methods of the present invention include, for example, those disclosed in: WO 98/42752; WO 00/37504; U.S. Pat. No. 6,207,156; Hurwitz et al. (1998) Proc. Natl. Acad. Sci. USA 95(17):10067-10071; Camacho et al. (2004) J. Clin. Oncology 22(145): Abstract No. 2505 (antibody CP-675206); and Mokyr et al. (1998) Cancer Res. 58:5301-5304. In certain embodiments, the methods of the instant invention comprise use of an anti-CTLA-4 antibody that is a human sequence antibody, preferably a monoclonal antibody and in another embodiment is monoclonal antibody 10D1. In another particular embodiment, the CTLA-4 inhibitor is ipilimumab or tremelimumab.

PD-1 (Programmed cell Death protein 1), also known as CD279, is a cell surface receptor expressed on activated T cells and B cells. Interaction with its ligands has been shown to attenuate T-cell responses both in vitro and in vivo. PD-1 binds two ligands, PD-L1 and PD-L2. PD-1 belongs to the immunoglobulin superfamily. PD-1 signaling requires binding to a PD-1 ligand in close proximity to a peptide antigen presented by major histocompatibility complex (MHC) (Freeman, Proc Natl Acad Sci USA 105, 10275-6 (2008)). Therefore, proteins, antibodies or small molecules that prevent co-ligation of PD-1 and TCR on the T cell membrane are useful PD-1 antagonists.

In one embodiment, the PD-1 receptor antagonist is an anti-PD-1 antibody, or an antigen binding fragment thereof, which specifically binds to PD-1 and blocks the binding of PD-L1 to PD-1. The anti-PD-1 antibody may be a monoclonal antibody. The anti-PD-1 antibody may be a human or humanised antibody. An anti-PD-1 antibody is an antibody capable of specific binding to the PD-1 receptor. Anti-PD-1 antibodies known in the art and suitable for the invention include nivolumab, pembrolizumab, pidilizumab, BMS-936559, and toripalimab.

PD-1 antagonists of the present invention also include compounds or agents that either bind to and/or block a ligand of PD-1 to interfere with or inhibit the binding of the ligand to the PD-1 receptor, or bind directly to and block the PD-1 receptor without inducing inhibitory signal transduction through the PD-1 receptor. In particular PD-1 antagonists include small molecules inhibitors of the PD-1/PD-L1 signalling pathway. Alternatively, the PD-1 receptor antagonist can bind directly to the PD-1 receptor without triggering inhibitory signal transduction and also binds to a ligand of the PD-1 receptor to reduce or inhibit the ligand from triggering signal transduction through the PD-1 receptor. By reducing the number and/or amount of ligands that bind to PD-1 receptor and trigger the transduction of an inhibitory signal, fewer cells are attenuated by the negative signal delivered by PD-1 signal transduction and a more robust immune response can be achieved.

In one embodiment, the PD-1 receptor antagonist is an anti-PD-L1 antibody, or an antigen binding fragment thereof, which specifically binds to PD-L1 and blocks the binding of PD-L1 to PD-1. The anti-PD-L1 antibody may be a monoclonal antibody. The anti-PD-L1 antibody may be a human or humanized antibody, such as atezolizumab (MPDL3280A) or avelumab.

Any aspect of the invention as described herein may be performed in combination with additional therapeutic agents, in particular additional cancer therapies. In particular, the hCCR8 binder and, optionally, the immune checkpoint inhibitor according to the present invention may be administered in combination with co-stimulatory antibodies, chemotherapy and/or radiotherapy (by applying irradiation externally to the body or by administering radio-conjugated compounds), cytokine-based therapy, targeted therapy, monoclonal antibody therapy, or any combination thereof.

A chemotherapeutic entity for combination therapy as used herein refers to an entity which is destructive to a cell, that is the entity reduces the viability of the cell. The chemotherapeutic entity may be a cytotoxic drug. A chemotherapeutic agent contemplated includes, without limitation, alkylating agents, anthracyclines, epothilones, nitrosoureas, ethylenimines/methylmelamine, alkyl sulfonates, alkylating agents, antimetabolites, pyrimidine analogs, epipodophylotoxins, enzymes such as L-asparaginase; biological response modifiers such asIFN-γ, IL-2, IL-12, and G-CSF; platinum coordination complexes such as cisplatin, oxaliplatin and carboplatin, anthracenediones, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

The additional cancer therapy may be other antibodies or small molecule reagents that reduce immune regulation in the periphery and within the tumour microenvironment, for example molecules that target TGFbeta pathways, IDO (indoleamine deoxigenase), Arginase, and/or CSF1R.

‘In combination’ or treatments comprising administration of a further therapeutic may refer to administration of the additional therapy before, at the same time as or after administration of any aspect according to the present invention. Combination treatments can thus be administered simultaneous, separate or sequential.

In another embodiment, the invention provides a kit comprising any of the binders as described above. In some embodiments, the kit further contains a pharmaceutically acceptable carrier or excipient. In other related embodiments, any of the components of the above combinations in the kit are present in a unit dose, in particular the dosages as described herein. In a yet further embodiment, the kit includes instructions for use in administering any of the components or the above combinations to a subject. In one particular embodiment, the kit comprises a hCCR8 binder as described herein and an immune checkpoint inhibitor, such as a PD-1 or PD-L1 inhibitor. The hCCR8 binder and the immune checkpoint inhibitor can be present in the same or in a different composition.

In one particular embodiment, the present invention provides a package comprising a binder as described herein, wherein the package further comprises a leaflet with instructions to administer the binder to a tumour patient that also receives treatment with an immune checkpoint inhibitor.

Diagnosis

The hCCR8 binder as described herein can further be used for predicting, diagnosing, prognosticating and/or monitoring diseases or conditions in subjects. In a particular embodiment, the invention provides a method for monitoring a cellular population expressing hCCR8 comprising contacting the cellular population with a hCCR8 binder of the invention.

In a further embodiment, the invention provides the use of an hCCR8 binder as described herein as a companion diagnostic in a method for treating a disease in a subject, wherein the treatment comprises administering a Treg depletion therapy. More in particular, wherein the treatment comprises administering a cytotoxic hCCR8 binder, such as an anti-hCCR8 antibody having ADCC activity, in particular an anti-hCCR8 antibody having ADCC activity and binding to the N-terminal region of hCCR8. Through its binding to the extracellular loops of hCCR8, the antibodies of the invention can be used to monitor treatment, e.g. monitor the depletion of Tregs, with an hCCR8 binder that binds to the N-terminal part of hCCR8.

As used herein, the terms “diagnosing” or “diagnosis” generally refer to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). As used herein, “diagnosis of a disease” in a subject may particularly mean that the subject has said disease, hence, is diagnosed as having said disease. A subject may be diagnosed as taught herein as not having said disease despite displaying one or more conventional symptoms or signs reminiscent thereof.

As used herein, the terms “prognosticating” or “prognosis” generally refer to an anticipation on the progression of a disease or condition and the prospect (e.g., the probability, duration, and/or extent) of recovery. A good prognosis of a disease may generally encompass anticipation of a satisfactory partial or complete recovery from said disease, preferably within an acceptable time period. A good prognosis of said disease may more commonly encompass anticipation of not further worsening or aggravating of the conditions, preferably within a given time period. A poor prognosis of a disease may generally encompass anticipation of a substandard recovery and/or unsatisfactorily slow recovery, or to substantially no recovery or even further worsening of said disease.

In one aspect, the present invention concerns a hCCR8 binder according to the invention for use in a method for diagnosing, predicating and/or prognosticating diseases associated with variations in the expression and/or activity of human CCR8. In other words, the invention provides an (in vitro) method for diagnosing predicating and/or prognosticating a disease associated with variations of the expression and/or the activity of CCR8 in a subject, wherein the method comprises measuring the quantity of CCR8 in a sample from the subject.

In a further aspect, the present invention concerns a hCCR8 binder according to the invention for use in a method for diagnosing, predicating and/or prognosticating diseases associated with variations in the expression and/or activity of human CCL1. In other words, the invention provides an (in vitro) method for diagnosing predicating and/or prognosticating a disease associated with variations of the expression and/or the activity of CLL1 in a subject, wherein the method comprises measuring the quantity of CCR8 in a sample from the subject.

According to another aspect, the invention concerns a kit for diagnosing, predicating and/or prognosticating a disease associated with variations of the expression and/or the activity of CCR8 and/or CCL1 comprising means for measuring the quantity of hCCR8 by using the hCCR8 binder as described herein. According to a preferred embodiment, said kit comprises a reference control obtained from a subject not suffering from said disease or having a known diagnosis, prediction and/or prognosis of said disease.

In an embodiment, the hCCR8 binder as described above may be advantageously immobilised on a solid phase or support.

Said kit can also comprise a known quantity or concentration of hCCR8 and/or a fragment thereof, e.g. for use as controls, standards and/or calibrators. It can also comprise means for collecting the sample from the subject.

An advantage of the binders of the invention, and in particular to the binders described herein that lack cytotoxic activity, is that they are suitable for diagnostic in vivo use. As they bind to the extracellular loops of CCR8 and in the absence of a cytotoxic moiety, the binders of the invention can be administered to a subject without influencing therapeutic treatment with non-competing CCR8 binders, such as N-terminal binders of the prior art. For example, the single-domain antibody moieties described herein, such as the VHH molecules specified herein before and in the examples, can be administered e.g. for imaging purposes while the patient undergoes treatment, such as with an anti-cancer drug, such as Treg depletion therapy. The non-cytotoxic binders can be used for imaging purposes, e.g. to monitor efficacious CCR8-expressing Treg depletion. Therefore, in a particular embodiment, the present invention provides a CCR8 binder comprising a CCR8 binding moiety as described herein and a detectable label. The detectable label may be detectable using e.g. radioactive, optical, magnetic resonance, and ultrasound approaches. In a particular embodiment, the detectable label is a fluorescent label. In a particular embodiment, the CCR8 binder of the invention, preferably lacking a cytotoxic moiety, is used for monitoring therapy with a non-competing CCR8 binder. In another embodiment, the CCR8 binder of the invention, preferably lacking a cytotoxic moiety, is used for monitoring therapy with an anti-CCR8 antibody that binds to the N-terminal region of hCCR8. In particular, the anti-CCR8 antibody that binds to the N-terminal region of hCCR8 is one of the antibodies disclosed in WO2020138489 A1, more in particular an anti-CCR8 antibody comprising a light chain variable region comprising SEQ ID NO:59 and heavy chain variable region comprising SEQ ID NO: 41 of WO2020138489 A1. In another embodiment, comprising the light chain constant region comprises SEQ ID NO: 52 and the heavy chain constant region comprises SEQ ID NO: 53 of WO2020138489 A1.

### EXAMPLES

The following examples are provided in order to demonstrate and further illustrate certain preferred embodiments and aspects of the present invention and are not construed as limiting the scope thereof.

Two anti-human CCR8 blocking monoclonal antibodies, ONCC8 and ONCC10 were used as control for the experiments described below. The sequence of ONCC8 was obtained by cloning the sequences of a light chain variable region and a heavy chain variable region from WO2020/0138489 A1 (corresponding respectively to SEQ ID NO: 59 and SEQ ID NO: 41 of WO2020/0138489 A1) into a human IgG1 backbone, whereas the sequence of ONCC10 was obtained by cloning the heavy chain and light chain variable region sequences from mAb 433H of WO2007/044756 A1 into a human IgG1 backbone. Production of these antibodies was performed in HEK293 cells by Icosagen (Icosagen, Tartu, Estonia) or in CHO cells by Evitria (Evitria, Zurich, Switzeland).

### Example 1. Generation of hCCR8-Targeting Single-Domain Antibody Moieties hCCR8 DNA Immunization

Immunization of llamas and alpacas with human CCR8 DNA was performed essentially as disclosed in Pardon E., et al. (A general protocol for the generation of Nanobodies for structural biology, Nature Protocols, 2014, 9(3), 674-693) and Henry K. A. and MacKenzie C. R. eds. (Single-Domain Antibodies: Biology, Engineering and Emerging Applications. Lausanne: Frontiers Media). Briefly, animals were immunized four times at two-week intervals with 2 mg of DNA encoding human CCR8 inserted into the expression vector pVAX1 (ThermoFisher Scientific Inc., V26020), after which blood samples were taken. Three months later, all animals received three injections of 2 mg of the same DNA, after which blood samples were taken.

Phage Display Library Preparation

Phage display libraries derived from peripheral blood mononuclear cells (PBMCs) were prepared and used as described in Pardon E., et al. (A general protocol for the generation of Nanobodies for structural biology, Nature Protocols, 2014, 9(3), 674-693) and Henry K. A. and MacKenzie C. R. eds. (Single-Domain Antibodies: Biology, Engineering and Emerging Applications. Lausanne: Frontiers Media). The VHH fragments were inserted into a M13 phagemid vector containing MYC and His6 tags. The libraries were rescued by infecting exponentially-growing Escherichia coli TG1 [(F′ traD36 proAB laclqZ ΔM15) supE thi-1 Δ(lac-proAB) Δ(mcrB-hsdSM)5(rK−mK−)] cells followed by surinfection with VCSM13 helper phage.

Phage display libraries were subjected to two consecutive selection rounds on HEK293T cells transiently transfected with human CCR8 inserted into pcDNA3.1 (ThermoFisher Scientific Inc., V79020) followed by CHO-K1 cells transiently transfected with human CCR8 inserted into pcDNA3.1. Polyclonal phagemid DNA was prepared from E. coli TG1 cells infected with the eluted phages from the second selection rounds. The VHH fragments were amplified by means of PCR from these samples and subcloned into an E. coli expression vector, in frame with N-terminal PeIB signal peptide and C-terminal FLAG3 and His6 tags. Electrocompetent E. coli TG1 cells were transformed with the resulting VHH-expression plasmid ligation mixture and individual colonies were grown in 96-deep-well plates. Monoclonal VHHs were expressed essentially as described in Pardon E., et al. (A general protocol for the generation of Nanobodies for structural biology, Nature Protocols, 2014, 9(3), 674-693). The crude periplasmic extracts containing the VHHs were prepared by freezing the bacterial pellets overnight followed by resuspension in PBS and centrifugation to remove cellular debris.

### Example 2. Generation of Stable CCR8 Cell Lines

Culturing of human embryonic kidney cell line HEK293 (ATTC N^{o} CRL-1573) was performed at 37° C. and 5% CO2 in Dulbecco's Modified Eagle Medium (DMDM, Gibco) supplemented with 10% heat-inactivated fetal bovine serum (FBS) and 100 U/ml penicillin and streptomycin (Gibco). Before transfection, cells were seeded at a density of 7.5×10 5 cells/well of 6-well plates (Greiner) and cultured overnight. Upon reaching an approximate confluence of 40%, cells were transfected with linearized pcDNA3.1 encoding human CCR8 using FUGENE HD transection reagent (Promega). After 6 hours, cellular supernatants were carefully removed and replaced by fresh complete DMEM. After 48 hours, culture medium was replaced to include 500 μg/ml G-418 (ThermoFisher Scientific Inc.) to select for gentamycin-resistant transfectants harbouring the expression cassette. Medium was changed every 2-3 days. After 3 weeks, limiting 1:2 dilutions were made starting from 10³ cells per well to obtain monoclonal lines. Identification of hCCR8-expressing monoclonal lines was based on acquiring 10⁴ cells in flow cytometry (Attune NxT, ThermoFisher Scientific Inc.) using a phycoerythrin-labelled mouse anti-hCCR8/CD198 IgG2a (Biolegend, clone N^{o} L263G8, 360603).

Another stable cell line was generated in a HEK293T (DSMZ, ACC 635) background with linearized pdDNA3.1/hygro carrying the Macaca CCR8 sequence, essentially as detailed above for human CCR8.

### Example 3. Screening for hCCR8 Selection Outputs

Recombinant cells expressing hCCR8 were recovered using cell dissociated non-enzymatic solution (Sigma Aldrich, C5914-100 mL) and resuspended to a final concentration of 1.0×10⁶ cells/ml in FACS buffer. Dilutions (1:5 in FACS buffer) of crude periplasmic extracts containing VHHs were incubated with mouse anti-FLAG biotinylated antibody (Sigma Aldrich, F9291-1MG) at 5 μg/ml in FACS buffer for 30 min with shaking at room temperature. Cell suspensions were distributed into 96-well v-bottom plates and incubated with the VHH/antibody mixture with one hour with shaking on ice. Binding of VHHs to cells was detected with streptavidin R-PE (Invitrogen, SA10044) at 1:400 dilution (0.18 μg/ml) in FACS buffer, incubated for 30 minutes in the dark with shaking on ice. Surface expression of human CCR8 on transiently transfected cell lines was confirmed by means of PE anti-human CCR8 (Biolegend, 360603) antibody at 2 μg/ml.

### Example 4. Purification and Evaluation of Monovalent VHHs

Synthetic DNA fragments encoding hCCR8-binding VHHs were subcloned into an E. coli expression vector under control of an IPTG-inducible lac promoter, in frame with N-terminal PeIB signal peptide for periplasmic compartment-targeting and C-terminal FLAG3 and His6 tags. Electrocompetent E. coli TG1 cells were transformed and the resulting clones were sequenced. VHH proteins were purified from these clones by IMAC chromatography followed by desalting, essentially as described in Pardon E., et al. (A general protocol for the generation of Nanobodies for structural biology, Nature Protocols, 2014, 9(3), 674-693).

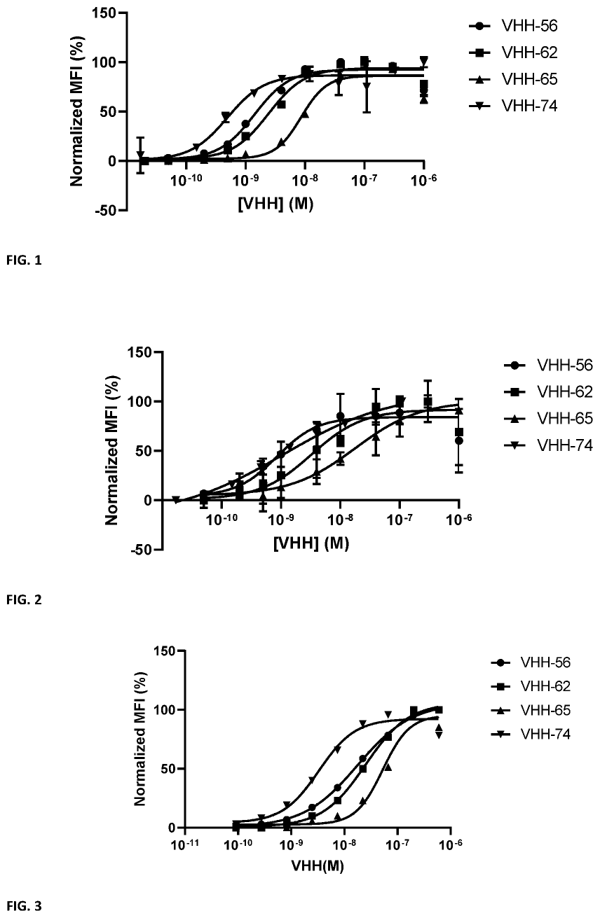

Eight purified VHHs obtained from the human CCR8 immunization campaign were selected and evaluated by flow cytometry for their binding to hCCR8. Four of the purified VHHs displayed potent binding to human CCR8 (FIG. 1).

### Example 5. In Vitro Cross-Reactivity Assessment of Monovalent VHHs

The eight purified VHHs obtained from the human CCR8 immunization campaign were further evaluated by flow cytometry for their binding to Macaca CCR8. For this purpose, both Macaca CCR8 stable cell lines (FIG. 2) and HEK293 cells transiently transfected with Macaca CCR8 (FIG. 3) were used. Four of the purified VHHs (VHH-56, VHH-62, VHH-65 and VHH-74, herein after) displayed potent binding to Macaca CCR8.

### Example 6. Epitope Mapping

VHH clones resulting from the human CCR8 immunization and selection campaign were screened by means of flow cytometry for binding to human CCR8 (SEQ ID NO: 79) on stably transfected HEK293 cells or to HEK293 cells previously transfected with plasmid DNA encoding three different versions of human-mouse chimera CCR8 in which one of the extracellular loops (ECLs) was replaced with the mouse CCR8 counterpart, as well as to an N-terminal deletion human CCR8 (SEQ ID NO: 80, delta 18-3XHA herein after), in comparison to mock-transfected control cells. Comparison of the binding (median fluorescent intensity) signal of a given VHH clone across the seven cell lines enabled classification of said clone as an N-terminal human CCR8 binder (i.e. binding on hCCR8 cells, but not on human CCR8 (delta18-3XHA) or control cells) or as an extracellular loop hCCR8 binder (i.e. binding on hCCR8 cells and on human CCR8 (delta18-3XHA), but not on control cells). Binding comparison across the cell lines expressing human-mouse chimera CCR8 allowed to further classify extracellular hCCR8 binders as either ECL1, ECL2 or ECL3 binders.

These experiments classified VHH-65 as an extracellular loop 2 (ECL2) human CCR8 binder, whereas VHH-74 competed with VHH-65 for CCR8 binding. VHH-62 was found to bind to both extracellular loops 2 and 3, whereas VHH-56 was classified as an extracellular loop 3 (ECL3) human CCR8 binder.

### Example 7. Binding and Functional Characterization of the Monovalent VHHs

The monovalent VHHs VHH-56, VHH-62, VHH-65 and VHH-74 were evaluated for their potential to functionally inhibit human CCL1 signalling on CHO-K1 cells displaying human CCR8 in cAMP accumulation experiments.

CHO-K1 cells stably expressing recombinant human CCR8 were grown prior to the test in media without antibiotic and detached by flushing with PBS-EDTA (5 mM EDTA), recovered by centrifugation and resuspended in KHR buffer (5 mM KCl, 1.25 mM MgSO₄, 124 mM NaCl, 25 mM HEPES, 13.3 mM Glucose, 1.25 mM KH₂PO₄, 1.45 mM CaCl₂), 0.5 g/l BSA, supplemented with 1 mM IBMX). Twelve microliters of cells were mixed with six microliters of VHH (final concentration: 1 μM) in triplicate and incubated for 30 minutes. Thereafter, six microliters of a mixture of forskolin and human CCL1 (R&D Systems, 845-TC or 272-I) was added at a final concentration corresponding to its EC80 value. The plates were then incubated for 30 min at room temperature. After addition of the lysis buffer and 1 hour incubation, fluorescence ratios were measured with the HTRF kit (Cisbio, 62AM9PE) according to the manufacturer's specification.

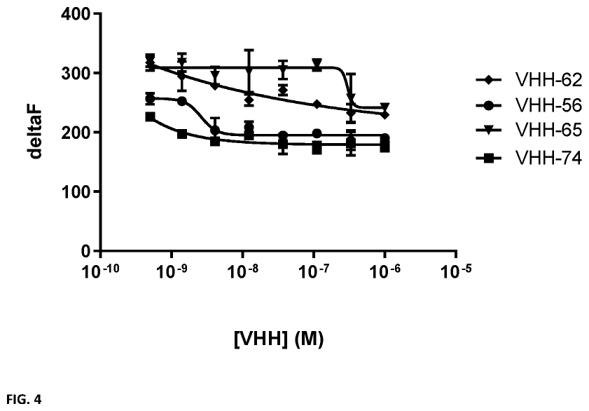

All of the tested VHHs provided potent functional inhibition in the assay with pIC50 values ranging from 6.75 to 9.74 M, indicating that these are blocking CCR8 binders (see FIG. 4).

### Example 8. Synthesis and Purification of Cross-Reactive VHH-Fc Fusions

In order to evaluate binding properties of VHH-Fc fusions of the hCCR8 binders, twelve VHH-Fc constructs (VHH-Fc-200, VHH-Fc-201, VHH-Fc-202, VHH-Fc-206, VHH-Fc-207, VHH-Fc-208, VHH-Fc-212, VHH-Fc-213, VHH-Fc-214, VHH-Fc-221, VHH-Fc-222 and VHH-Fc-223) were generated by combining anti-CCR8 VHHs to a human short hinge and IgG1 Fc domain (SEQ ID NO: 78), either by direct fusion (VHH-Fc-200, VHH-Fc-206, VHH-Fc-212 and VHH-Fc-221), or separated by flexible GlySer linkers 10GS (VHH-Fc-201, VHH-Fc-207, VHH-Fc-213 and VHH-Fc-222) or 20GS (VHH-Fc-202, VHH-Fc-208, VHH-Fc-214 and VHH-Fc-223) (20GS referring to four repeats of SEQ ID NO: 50 and, thus, having 20 amino acids in length). Constructs VHH-Fc-200, VHH-Fc-201 and VHH-Fc-202 contain a VHH-56 CCR8 binding moiety, VHH-Fc-206, VHH-Fc-207 and VHH-Fc-208 contain a VHH-62 CCR8 binding moiety, VHH-Fc-212, VHH-Fc-213 and VHH-Fc-214 contain a VHH-65 CCR8 binding moiety, whereas VHH-Fc-221, VHH-Fc-222 and VHH-Fc-223 contain a VHH-74 CCR8 binding moiety.

The constructs were cloned in a pQMCF mammalian expression vector, in frame with a secretory signal peptide to direct the expressed recombinant proteins to the extracellular environment. Cloning, cell transfection protein production in CHOEBNALT854 1E9 cells and protein A purification were performed by Icosagen (Icosagen Cell Factory, Tartu, Estonia).

### Example 9. Confirmation of hCCR8 Binding by VHH-Fc Fusions

The twelve multivalent VHH-Fc fusions were evaluated for their ability to bind to human CCR8 on stably transfected HEK293 cells by means of flow cytometry experiments. Cells were incubated with different concentrations of the multivalent VHH-Fc fusions for 30 minutes at 4° C., followed by two washes with FACS buffer, followed by 30 minutes incubation at 4° C. with R-Phycoerythrin AffiniPure F(ab′) 2 Fragment Goat Anti-human IgG (Jackson ImmunoResearch, cat. #109-116-098), followed by two washing steps. Dead cells were stained using TOPRO3 (Thermo Fisher Scientific, T3605).

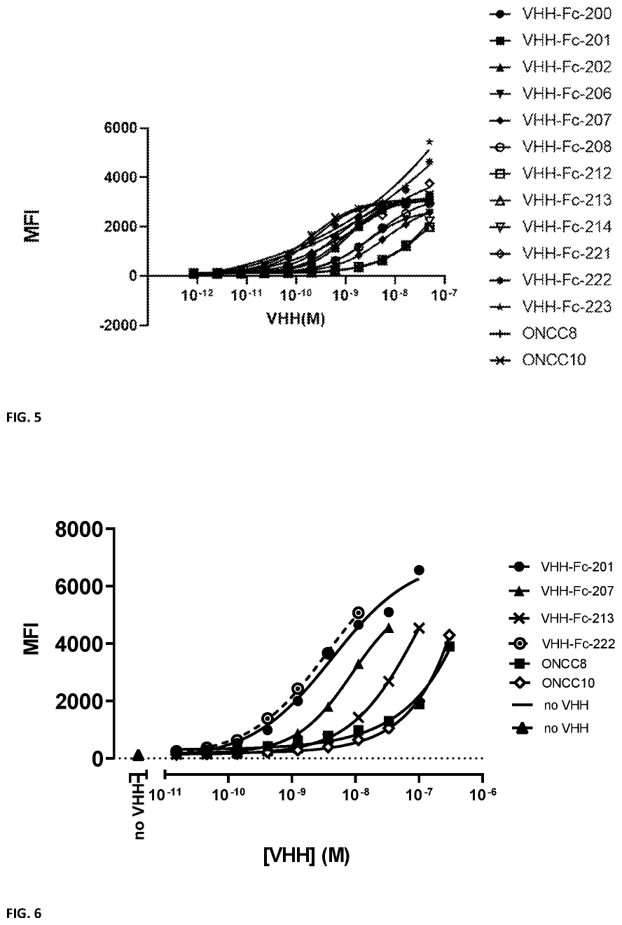

The binding of all twelve VHH-Fc fusions to human CCR8 was highly comparable, with pEC50 values ranging from 7.64 to 9.31 M (FIG. 5).

### Example 10. In Vitro Cross-Reactivity Assessment of VHH-FC Fusions

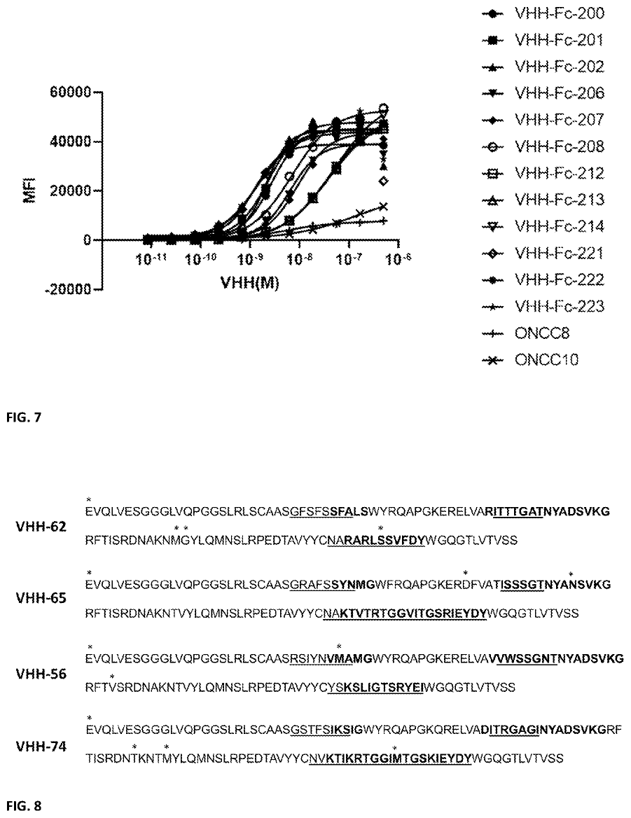

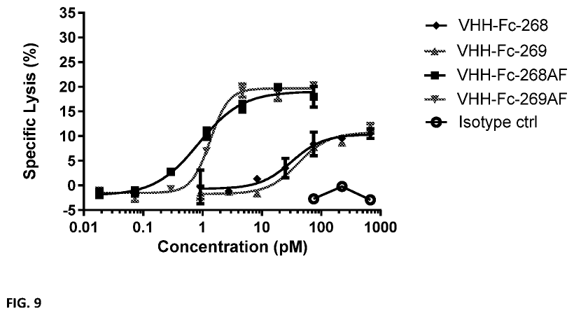

All twelve multivalent VHH-Fc fusions were evaluated for their ability to bind to Macaca CCR8 transiently expressed in HEK293 cells (FIG. 6) by means of flow cytometry experiments. Similar experiments were also performed on a for a selection of VHH-Fc fusions on HEK293T cells stably expressing Macaca CCR8 (FIG. 7). Cells were incubated with different concentrations of the multivalent VHH-Fc fusions for 30 minutes at 4° C., followed by two washes with FACS buffer, followed by 30 minutes incubation at 4° C. with AF488 goat anti-mouse IgG (Life Technologies, A11029) or AF488 donkey anti-rat IgG (Life Technologies, A21208), followed by two washing steps. Dead cells were stained using TOPRO3 (Thermo Fisher Scientific, T3605).

The binding of all VHH-Fc fusions to Macaca CCR8 were found to be highly comparable, with pEC50 values ranging from 6.98 to 8.93 M, confirming that all twelve VHH-Fc are cross-reactive with Macaca CCR8 with EC values comparable to human CCR8.

### Example 11. ADCC Potency of VHH-Fc Fusions

ADCC Reporter Gene Assay

All twelve VHH-FC fusions were tested for their capacity to activate human FcγRIIIa in the ADCC reported assay (Promega, G7010, G7018) using human CCR8 HEK293 cell line as target cells.

Engineered Jurkat cells stably transfected with the V158 FcγRIIIa receptor and an NFAT (nuclear factor of activated T-cells) responsive firefly luciferase reporter gene as effector cells were used in this assay. HEK293 cells overexpressing human CCR8 were used as target cells. ADCC activity was quantified through the produced luciferase luminescence signal resulting from the NFAT pathway activation upon incubation of the VHH-Fc fusions with the target and effector cells at a 2.5:1 effector:target cell ratio, according to the recommendations of the manufacturer.

All six VHH-Fc fusions were found to activate the human FcγRIIIa with pEC50 values ranging from approximately 7.81 to 9.45 M, based on four dilutions, in the same range as the two control anti-human CCR8 antibodies ONCC8 and ONCC10.

ADCC Assay Using Human PBMC

Three VHH-Fc fusions (VHH-Fc-221, VHH-Fc-222 and VHH-Fc-223) were tested along with control monoclonal antibody ONCC8 and an isotype control in the ADCC assay using human PBMC from three independent healthy donors in a 40:1 effector:target ratio. Briefly, HEK293 cells expressing human CCR8 were labelled with DiO and seeded in 96-well round bottom plates at 5×10³ cells per well. Binders were subjected to an 8-point titration in duplicate. Labelled target cells were opsonized with titration of the binders followed by incubated with effector cells for 3 hours. Specific lysis on target cells was monitored by the PI live/dead stain. Samples were acquired on a NxT flow cytometer (Attune).

All twelve VHH-Fc fusions displayed potent ADCC activity with pEC50 values ranging from approximately 10.5 to 11.8 M, based on the average of three independent experiments using human PBMC from different healthy donors, within the same range as the ONCC8 control.

### Example 12. Sequence Optimization of Monovalent VHHs

VHH-56, VHH-62, VHH-65 and VHH-74 were subjected to sequence optimization in an attempt to maximally improve its sequence in terms of humanization towards human IGHV3 (SEQ ID NO: 93, EVQLVESGGGLVQPGGSLRLSCAASGFTFSSYAMHWVRQAPGKGLEWVSVISSDGSSTYY ADSVKGRFTISRDNSKNTLYLQMNSLRAEDTAVYYCAR) and JH (SEQ ID NO: 94, WGQGTLVTVSS) germline consensus sequences, as well as in terms of chemical and biophysical stability,

Production, Purification and Assessment of the Binding Properties

Cloning into an E. coli expression vector with a C-terminal His6 (but no FLAG3) tag, standard E. coli expression and immobilized metal-affinity chromatography (IMAC) steps were carried out as described in Example 4.

Multiple His6 tagged VHH variants were thus generated and evaluated by flow cytometry f for their ability to compete for binding to hCCR8 with their FLAG3-His6 tagged parental counterparts. Cells were first incubated with different concentrations of monovalent sequence optimization variants that do not carry a FLAG3 tag for 30 minutes at 4° C., followed by a 30 minutes incubation at 4° C. of a fixed concentration of FLAG3-tagged parental VHH, followed by washing and anti-FLAG detection by means of mouse M2 anti-Flag mAb (Sigma Aldrich, cat. #F-1804) followed by R-Phycoerythrin AffiniPure F(ab′)₂ Fragment Goat Anti-Mouse IgG (Jackson ImmunoResearch, cat. #115-116-071). Variants that retained binding capacity where selected for further biophysical and chemical stability analysis.

Biophysical Stability

All VHH-56, VHH-62, VHH-65 and VHH-74 variants as well as their Fc-fusions were subjected to thermal stability and aggregation assays to gain insights in their melting and aggregation temperatures.

Intrinsic tryptophan-fluorescence was monitored upon temperature-induced protein unfolding in an Unlce instrument (Unchained Labs, Pleasanton, CA, USA). Ten microliter samples were applied at 1 mg/ml to the sample cuvette, and a linear temperature ramp was initiated from 25 to 95° C. at a rate of 0.5° C. per minute, with a pre-run of 180 seconds. The barycentric mean (BCM) and static light scattering (SLS at 266 nm and 473 nm) signals were plotted against temperature in order to obtain melting temperatures (Tₘ) and aggregation temperatures (T_{agg}), respectively.

When subjecting the VHH samples to temperature-induced unfolding, clear differences in melting temperatures (Tₘ) could be noted which were well corroborated by aggregation temperatures (T_{agg}) determined by static light scattering at 266 nm (smaller aggregates) and 473 nm (larger aggregates).

Dynamic light scattering was performed using the Uncle instrument by applying 10 μl of sample at 1 mg/ml to the sample cuvette. Laser and attenuator controls were set at Auto while 10 acquisitions were run per data point with an acquisition time of 10 seconds each.

Size-exclusion chromatography (SEC) coupled to multi-angle laser-light scattering (MALLS) was carried out by applying 120 μl of a 1 mg/ml sample to a Superdex 200 column (GE healthcare) on an Agilent HPLC system. The outlet of the column was coupled to a UV detector followed by refractive index (RI) detection and finally MALLS detector.

SEC-MALLS data for the VHH variants and their Fc-fused counterparts nicely correlated. For the majority of the VHH-Fc fusions containing the VHH entities, storage at 40° C. for one week did not lead to any observable liabilities in terms of (in)soluble (SEC-MALLS) aggregates being present.

Chemical Stability

Purified VHHs obtained from the sequence optimization campaign of were selected for chemical stability assessment.

The following binders were selected:
VHH-81: VHH-62 E1D, M77T, G78V, S103W
VHH-89: VHH-65 E1D, D46E, N60D
VHH-114: VHH-56 E1D, M32L, V691
VHH-115: VHH-56 E1D, M32R, V691
VHH-125: VHH-74 E1D, T74A, M78V, M107L

Samples were stored at 40° C. for 4 weeks, whereas reference samples were stored at −80° C. Forced oxidized samples (at 1 mg/ml) were supplemented with hydrogen peroxide up to a final concentration of 10 mM, followed by incubation at 37° C. for three hours, with final buffer exchange to phosphate buffer saline (PBS) using PD MidiTrap G-25 columns (GE-Healthcare, Chicago, IL, USA) according to instructions of the manufacturer. Samples were stored at −80° C. until lass spectrometric peptide mapping (Research Institute for Chromatography, Kortrijk, Belgium). Peptide mapping consisted in treating 100 μg of the sample proteins with trypsin (overnight at 25° C.) and injecting the samples onto an RPC-column (reversed phase chromatography; elution by applying an acetonitrile gradient) followed by the ESI-mass spectrometer where LC-MS and LC-MS/MS data were used for quantification and identification, respectively.

It was found that the E1D mutation in all of VHH-56, VHH-62, VHH-65 and VHH-74 eliminated pyroglutamatic acid formation. Furthermore, substituting a methionine residue by either a leucine or an arginine in position 32 of VHH-56 improved expression levels of VHH-114 and VHH-115 in E. coli compared to VHH-56(E1D) and VHH-56(E1D,V691) controls. In addition, the combination of M32L or M32R with V691 improved thermal stability by respectively 5° C. and 4° C. compared to parental VHH-56. The combined introduction of G78V and M77T mutations improved thermal stability by 5° C. compared to parental VHH-62 and the additional introduction of S103W further improved thermal stability by another 6° C. The threonine to alanine substitution in position 74 of VHH-74 improved thermal stability by 8° C. Interestingly, substituting a methionine residue by a leucine at position 107 of VHH-74 considerably reduced levels of forced oxidation, which was as high as 72% in non-optimized VHH-74. Substitution of an aspartic acid to glutamic acid at position 46 of VHH-65 improved the thermal stability by 6° C. The other amino acid substitutions described above resulted in an increased humanization of the binders.

### Example 12. Synthesis and Purification of Optimized VHH-Fc Fusions

Fc-fusions of the optimized VHH sequences were generated as in example 8. Consequently, VHH-81 was fused directly to an IgG1 short hinge domain (SEQ ID NO: 55) or through a 10GS linker (SEQ ID NO: 64) or 20GS linker (SEQ ID NO: 73). Similarly, VHH-89 was fused directly to an IgG1 short hinge domain (SEQ ID NO: 56) or through a 10GS linker (SEQ ID NO: 65) or 20GS linker (SEQ ID NO: 74). VHH-114 was fused directly to an IgG1 short hinge domain (SEQ ID NO: 57) or through a 10GS linker (SEQ ID NO: 66) or 20GS linker (SEQ ID NO: 75). VHH-115 was fused directly to an IgG1 short hinge domain (SEQ ID NO: 58) or through a 10GS linker (SEQ ID NO: 67) or 20GS linker (SEQ ID NO: 76). VHH-125 was fused directly to an IgG1 short hinge domain (SEQ ID NO: 59) or through a 10GS linker (SEQ ID NO: 68) or 20GS linker (SEQ ID NO: 77). These constructs retained binding capacity are optimally suited for treatment of the diseases mentioned herein.

### Example 13. ADCC Potency of Optimized VHH-Fc Fusions

Two of the Fc-fusions of the optimized sequences obtained in Example 12 (VHH-125 fused either directly to an IgG1 short hinge domain (SEQ ID NO: 59, referred to hereinafter as VHH-Fc-268) or through a 10GS linker (SEQ ID NO: 68, referred to hereinafter as VHH-Fc-269)) were tested along with an isotype control in the ADCC assay using human PBMC from three independent healthy donors in a 40:1 effector:target ratio. The ADCC potency was assessed for both the afucosylated and non-afucosylated versions of the VHH-Fc fusions.

Briefly, HEK293 cells expressing human CCR8 were labelled with DiO and seeded in 96-well round bottom plates at 5×10³ cells per well. Binders were subjected to an 8-point titration in duplicate. Labelled target cells were opsonized with titration of the binders followed by incubated with effector cells for 3 hours. Specific lysis on target cells was monitored by the PI live/dead stain. Samples were acquired on a NxT flow cytometer (Attune).

Both the afucosylated and non-afucosylated versions of the VHH-Fc fusions displayed potent ADCC activity in comparison with the isotype control (see FIG. 19). The observed ADCC activity of the afucosylated version of VHH-Fc fusions displayed the strongest ADCC activity. These data show that the Fc-fusions of the optimized VHH sequences show potent ADCC activity, while the afucosylated version of said Fc-fusions performs even better.

1. A human CCR8 (hCCR8) binder, wherein the binder comprises three complementary determining regions (CDRs), CDR1, CDR2 and CDR3, in which CDR3 is selected from the group consisting of
a) the amino acid sequence of NARARLWSVFDY (SEQ ID NO: 10);
b) the amino acid sequence of NAKTVTRTGGVITGSRIEYDY (SEQ ID NO: 11);
c) the amino acid sequence of YSKSLIGTSRYEI (SEQ ID NO: 12);
d) the amino acid sequence of NVKTIKRTGGILTGSKIEYDY (SEQ ID NO: 13);
e) amino acid sequences having at least 80% amino acid identity with at least one of the sequences of SEQ ID NO: 10⁻¹³; and
f) amino acid sequences having 3, 2 or 1 amino acid sequence difference with at least one of the sequences of SEQ ID NO: 10-13.

2. The binder of claim 1, wherein
CDR1 is selected from the group consisting of
a) the amino acid sequence of GFSFSSFA (SEQ ID NO: 1);
b) the amino acid sequence of GRAFSSYN (SEQ ID NO: 2);
c) the amino acid sequence of RSIYNVLA (SEQ ID NO: 3);
d) the amino acid sequence of RSIYNVRA (SEQ ID NO: 4);
e) the amino acid sequence of GSTFSIKS (SEQ ID NO: 5);
f) amino acid sequences having at least 80% amino acid identity with at least one of the sequences of SEQ ID NO: 1-5; and
g) amino acid sequences having 3, 2, 1 amino acid difference with at least one of the sequences of SEQ ID NO: 1-5;
and
CDR2 is selected from the group consisting of
h) the amino acid sequence of
(SEQ ID NO: 6)
ITTTGAT;
i) the amino acid sequence of
(SEQ ID NO: 7)
ISSSGT;
j) the amino acid sequence of
(SEQ ID NO: 8)
VWSSGNT;
k) the amino acid sequence of
(SEQ ID NO: 9)
ITRGAGI;
l) amino acid sequences having at least 80% amino acid identity with at least one of the sequences of SEQ ID NO: 6-9; and
m) amino acid sequences having 3, 2, 1 amino acid difference with at least one of the sequences of SEQ ID NO: 6-9;
and
CDR3 is selected from the group consisting of
n) the amino acid sequence of
(SEQ ID NO: 10)
NARARLWSVFDY;
o) the amino acid sequence of
(SEQ ID NO: 11)
NAKTVTRTGGVITGSRIEYDY;
p) the amino acid sequence of
(SEQ ID NO: 12)
YSKSLIGTSRYEI;
q) the amino acid sequence of
(SEQ ID NO: 13)
NVKTIKRTGGILTGSKIEYDY;
r) amino acid sequences having at least 80% amino acid identity with at least one of the sequences of SEQ ID NO: 10-13; and
s) amino acid sequences having 3, 2 or 1 amino acid sequence difference with at least one of the sequences of SEQ ID NO: 10-13.

3. The binder of claim 1, wherein the single-domain antibody moiety further comprises four framework regions (FRs), FR1, FR2, FR3 and FR4, wherein
FR1 has at least 85% sequence identity to SEQ ID NO: 14;
FR2 has at least 85% sequence identity to SEQ ID NO: 15, SED ID NO: 16, SEQ ID NO: 17 or SEQ ID NO: 18;
FR3 has at least 85% sequence identity to SEQ ID NO: 19;
FR4 has at least 85% sequence identity to SEQ ID NO: 20.

4. The binder of claim 1, wherein the single-domain antibody moiety comprises the amino acid sequence of SEQ ID NO: 21, or SEQ ID NO: 22, or SEQ ID NO: 23, or SEQ ID NO: 24, or SEQ ID NO: 25.

5. The binder of claim 1, wherein the binder is cross-reactive with non-human primate CCR8.

6. The binder of claim 1, wherein the binder further comprises a cytotoxic moiety.

7. The binder of claim 6, wherein the cytotoxic moiety
induces antibody-dependent cellular cytotoxicity (ADCC),
induces complement-dependent cytotoxicity (CDC),
induces antibody-dependent cellular phagocytosis (ADCP),
binds to and activates T-cells, or
comprises a cytotoxic payload.

8. The binder of claim 6, wherein the cytotoxic moiety comprises a fragment crystallisable (Fc) region moiety.

9. The binder of claim 8, wherein the Fc region moiety has been engineered to increase ADCC, CDC, and/or ADCP activity, such as through afucosylation or by comprising an ADCC, CDC and/or ADCP-increasing mutation.

10. (canceled)

11. The binder of claim 1, wherein the single-domain antibody moiety is comprised in a medicine.

12. A method for the treatment of a tumour in a subject, the method comprising: administering to the subject the binder of claim 1.

13. The method according to claim 12, wherein the tumour is selected from the group consisting of a breast cancer, uterine corpus cancer, lung cancer, stomach cancer, head and neck squamous cell carcinoma, skin cancer, colorectal cancer, kidney cancer and T cell lymphoma.

14. The method according to claim 12, wherein administration of the binder depletes tumour-infiltrating regulatory T-cells (Tregs) in the subject.

15. The binder or the nucleic acid for use according to any one of claims 11 to 14 method according to claim 12, wherein the method further comprises administration of a checkpoint inhibitor, or an inhibitor that blocks PD-1 or PD-L1.

16. A human CCR8 (hCCR8) binder, wherein said binder comprises three complementary determining regions (CDRs), CDR1, CDR2 and CDR3, wherein the CDRs are selected from the respective CDR1s, CDR2s, and CDR3s comprised in SEQ ID SEQ ID NO: 21, 22, 23, 24 and 25; wherein the CDRs are defined by the Kabat, Chothia, AHo or international ImMunoGeneTics (IMGT) information system.


SEQUENCE LISTING (94 sequences)

SEQ ID NO: 1 - protein, 8 aa, Artificial sequence
  CDR1-IMGT-P62/P81
GFSFSSFA

SEQ ID NO: 2 - protein, 8 aa, Artificial sequence
  CDR1-IMGT-P65/P89
GRAFSSYN

SEQ ID NO: 3 - protein, 8 aa, Artificial sequence
  CDR1-IMGT-P114
RSIYNVLA

SEQ ID NO: 4 - protein, 8 aa, Artificial sequence
  CDR1-IMGT-P115
RSIYNVRA

SEQ ID NO: 5 - protein, 8 aa, Artificial sequence
  CDR1-IMGT-P74/P125
GSTFSIKS

SEQ ID NO: 6 - protein, 7 aa, Artificial sequence
  CDR2-IMGT-P62/P81
ITTTGAT

SEQ ID NO: 7 - protein, 6 aa, Artificial sequence
  CDR2-IMGT-P65/P89
ISSSGT

SEQ ID NO: 8 - protein, 7 aa, Artificial sequence
  CDR2-IMGT-P56/P114/P115
VWSSGNT

SEQ ID NO: 9 - protein, 7 aa, Artificial sequence
  CDR2-IMGT-P74/P125
ITRGAGI

SEQ ID NO: 10 - protein, 12 aa, Artificial sequence
  CDR3-IMGT-Kabat-P81
NARARLWSVFDY

SEQ ID NO: 11 - protein, 21 aa, Artificial sequence
  CDR3-IMGT-Kabat-P65/P89
NAKTVTRTGGVITGSRIEYDY

SEQ ID NO: 12 - protein, 13 aa, Artificial sequence
  CDR3-IMGT-Kabat-P56/P114/P115
YSKSLIGTSRYEI

SEQ ID NO: 13 - protein, 21 aa, Artificial sequence
  CDR3-IMGT-Kabat-P125
NVKTIKRTGGILTGSKIEYDY

SEQ ID NO: 14 - protein, 25 aa, Artificial sequence
  FR1-P81/P89/P114/P115/P125
DVQLVESGGGLVQPGGSLRLSCAAS

SEQ ID NO: 15 - protein, 17 aa, Artificial sequence
  FR2-P56/P114/P115
MGWYRQAPGKERELVAV

SEQ ID NO: 16 - protein, 17 aa, Artificial sequence
  FR2-P62/P81
LSWYRQAPGKERELVAR

SEQ ID NO: 17 - protein, 17 aa, Artificial sequence
  FR2-P89
MGWFRQAPGKEREFVAT

SEQ ID NO: 18 - protein, 16 aa, Artificial sequence
  FR2-P74/P125
GWYRQAPGKQRELVAD

SEQ ID NO: 19 - protein, 38 aa, Artificial sequence
  FR3-P81/P89/P114/P115/P125
NYADSVKGRFTISRDNAKNTVYLQMNSLRPEDTAVYYC

SEQ ID NO: 20 - protein, 11 aa, Artificial sequence
  FR4-P62/P65/P56/P74/P81/P89/P114/P115/P125
WGQGTLVTVSS

SEQ ID NO: 21 - protein, 118 aa, Artificial sequence
  VHH-P81
DVQLVESGGGLVQPGGSLRLSCAASGFSFSSFALSWYRQAPGKERELVARITTTGATNYADSVKGRFTISRDNAKNTVYLQMNSLRPEDTAVYYCNARARLWSVFDYWGQGTLVTVSS

SEQ ID NO: 22 - protein, 126 aa, Artificial sequence
  VHH-P89
DVQLVESGGGLVQPGGSLRLSCAASGRAFSSYNMGWFRQAPGKEREFVATISSSGTNYADSVKGRFTISRDNAKNTVYLQMNSLRPEDTAVYYCNAKTVTRTGGVITGSRIEYDYWGQGTLVTVSS

SEQ ID NO: 23 - protein, 119 aa, Artificial sequence
  VHH-P114
DVQLVESGGGLVQPGGSLRLSCAASRSIYNVLAMGWYRQAPGKERELVAVVWSSGNTNYADSVKGRFTISRDNAKNTVYLQMNSLRPEDTAVYYCYSKSLIGTSRYEIWGQGTLVTVSS

SEQ ID NO: 24 - protein, 119 aa, Artificial sequece
  VHH-P115
DVQLVESGGGLVQPGGSLRLSCAASRSIYNVRAMGWYRQAPGKERELVAVVWSSGNTNYADSVKGRFTISRDNAKNTVYLQMNSLRPEDTAVYYCYSKSLIGTSRYEIWGQGTLVTVSS

SEQ ID NO: 25 - protein, 127 aa, Artificial sequence
  VHH-P125
DVQLVESGGGLVQPGGSLRLSCAASGSTFSIKSIGWYRQAPGKQRELVADITRGAGINYADSVKGRFTISRDNAKNTVYLQMNSLRPEDTAVYYCNVKTIKRTGGILTGSKIEYDYWGQGTLVTVSS

SEQ ID NO: 26 - protein, 8 aa, Artificial sequence
  CDR1-IMGT-P56
RSIYNVMA

SEQ ID NO: 27 - protein, 12 aa, Artificial sequence
  CDR3-IMGT-P62
NARARLSSVFDY

SEQ ID NO: 28 - protein, 21 aa, Artificial sequence
  CDR3-IMGT-P74
NVKTIKRTGGIMTGSKIEYDY

SEQ ID NO: 29 - protein, 25 aa, Artificial sequence [sequence omitted]

SEQ ID NO: 30 - protein, 17 aa, Artificial sequence
  FR2-P65
MGWFRQAPGKERDFVAT

SEQ ID NO: 31 - protein, 38 aa, Artificial sequence
  FR3-P62
NYADSVKGRFTISRDNAKNMGYLQMNSLRPEDTAVYYC

SEQ ID NO: 32 - protein, 38 aa, Artificial sequence
  FR3-P65
NYANSVKGRFTISRDNAKNTVYLQMNSLRPEDTAVYYC

SEQ ID NO: 33 - protein, 38 aa, Artificial sequence
  FR3-P56
NYADSVKGRFTVSRDNAKNTVYLQMNSLRPEDTAVYYC

SEQ ID NO: 34 - protein, 38 aa, Artificial sequence
  FR3-P74
NYADSVKGRFTISRDNTKNTMYLQMNSLRPEDTAVYYC

SEQ ID NO: 35 - protein, 118 aa, Artificial sequence
  VHH-P62
EVQLVESGGGLVQPGGSLRLSCAASGFSFSSFALSWYRQAPGKERELVARITTTGATNYADSVKGRFTISRDNAKNMGYLQMNSLRPEDTAVYYCNARARLSSVFDYWGQGTLVTVSS

SEQ ID NO: 36 - protein, 126 aa, Artificial sequence
  VHH-P65
EVQLVESGGGLVQPGGSLRLSCAASGRAFSSYNMGWFRQAPGKERDFVATISSSGTNYANSVKGRFTISRDNAKNTVYLQMNSLRPEDTAVYYCNAKTVTRTGGVITGSRIEYDYWGQGTLVTVSS

SEQ ID NO: 37 - protein, 119 aa, Artificial sequence
  VHH-P56
EVQLVESGGGLVQPGGSLRLSCAASRSIYNVMAMGWYRQAPGKERELVAVVWSSGNTNYADSVKGRFTVSRDNAKNTVYLQMNSLRPEDTAVYYCYSKSLIGTSRYEIWGQGTLVTVSS

SEQ ID NO: 38 - protein, 127 aa, Artificial sequence
  VHH-P74
EVQLVESGGGLVQPGGSLRLSCAASGSTFSIKSIGWYRQAPGKQRELVADITRGAGINYADSVKGRFTISRDNTKNTMYLQMNSLRPEDTAVYYCNVKTIKRTGGIMTGSKIEYDYWGQGTLVTVSS

SEQ ID NO: 39 - protein, 10 aa, Artificial sequence
  CDR1-Kabat-P62/P81
GFSFSSFALS

SEQ ID NO: 40 - protein, 10 aa, Artificial sequence
  CDR1-Kabat-P65/P89
GRAFSSYNMG

SEQ ID NO: 41 - protein, 10 aa, Artificial sequence
  CDR1-Kabat-P56
RSIYNVMAMG

SEQ ID NO: 42 - protein, 10 aa, Artificial sequence
  CDR1-Kabat-P74/P125
GSTFSIKSIG

SEQ ID NO: 43 - protein, 16 aa, Artificial sequence
  CDR2-Kabat-P62/P81
RITTTGATNYADSVKG

SEQ ID NO: 44 - protein, 15 aa, Artificial sequence
  CDR2-Kabat-P65
TISSSGTNYANSVKG

SEQ ID NO: 45 - protein, 16 aa, Artificial sequence
  CDR2-Kabat-P56/P114/P115
VVWSSGNTNYADSVKG

SEQ ID NO: 46 - protein, 16 aa, Artificial sequence
  CDR2-Kabat-P74/P125
DITRGAGINYADSVKG

SEQ ID NO: 47 - protein, 10 aa, Artificial sequence
  CDR1-Kabat-P114
RSIYNVLAMG

SEQ ID NO: 48 - protein, 10 aa, Artificial sequence
  CDR1-Kabat-P115
RSIYNVRAMG

SEQ ID NO: 49 - protein, 15 aa, Artificial sequence
  CDR2-Kabat-P89
TISSSGTNYADSVKG

SEQ ID NO: 50 - protein, 5 aa, Artificial sequence
  GS linker
GGGGS

SEQ ID NO: 51 - protein, 344 aa, Artificial sequence
  P62-Fc
EVQLVESGGGLVQPGGSLRLSCAASGFSFSSFALSWYRQAPGKERELVARITTTGATNYADSVKGRFTISRDNAKNMGYLQMNSLRPEDTAVYYCNARARLWSVFDYSGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 352 aa, Artificial sequence
  P65-Fc
EVQLVESGGGLVQPGGSLRLSCAASGRAFSSYNMGWFRQAPGKERDFVATISSSGTNYANSVKGRFTISRDNAKNTVYLQMNSLRPEDTAVYYCNAKTVTRTGGVITGSRIEYDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 345 aa, Artificial sequence
  P56-Fc
EVQLVESGGGLVQPGGSLRLSCAASRSIYNVMAMGWYRQAPGKERELVAVVWSSGNTNYADSVKGRFTVSRDNAKNTVYLQMNSLRPEDTAVYYCYSKSLIGTSRYEIWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 353 aa, Artificial sequence
  P74-Fc
EVQLVESGGGLVQPGGSLRLSCAASGSTFSIKSIGWYRQAPGKQRELVADITRGAGINYADSVKGRFTISRDNTKNTMYLQMNSLRPEDTAVYYCNVKTIKRTGGIMTGSKIEYDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 344 aa, Artificial sequence
  P81-Fc
DVQLVESGGGLVQPGGSLRLSCAASGFSFSSFALSWYRQAPGKERELVARITTTGATNYADSVKGRFTISRDNAKNTVYLQMNSLRPEDTAVYYCNARARLWSVFDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 352 aa, Artificial sequence
  P89-Fc
DVQLVESGGGLVQPGGSLRLSCAASGRAFSSYNMGWFRQAPGKEREFVATISSSGTNYADSVKGRFTISRDNAKNTVYLQMNSLRPEDTAVYYCNAKTVTRTGGVITGSRIEYDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 345 aa, Artificial sequence
  P114-Fc
DVQLVESGGGLVQPGGSLRLSCAASRSIYNVLAMGWYRQAPGKERELVAVVWSSGNTNYADSVKGRFTISRDNAKNTVYLQMNSLRPEDTAVYYCYSKSLIGTSRYEIWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 345 aa, Artificial sequence
  P115-Fc
DVQLVESGGGLVQPGGSLRLSCAASRSIYNVRAMGWYRQAPGKERELVAVVWSSGNTNYADSVKGRFTISRDNAKNTVYLQMNSLRPEDTAVYYCYSKSLIGTSRYEIWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 353 aa, Artificial sequence
  P125-Fc
DVQLVESGGGLVQPGGSLRLSCAASGSTFSIKSIGWYRQAPGKQRELVADITRGAGINYADSVKGRFTISRDNAKNTVYLQMNSLRPEDTAVYYCNVKTIKRTGGILTGSKIEYDYWGQGTLVTVSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 354 aa, Artificial sequence
  P62-10GS-Fc
EVQLVESGGGLVQPGGSLRLSCAASGFSFSSFALSWYRQAPGKERELVARITTTGATNYADSVKGRFTISRDNAKNMGYLQMNSLRPEDTAVYYCNARARLWSVFDYSGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 362 aa, Artificial sequence
  P65-10GS-Fc
EVQLVESGGGLVQPGGSLRLSCAASGRAFSSYNMGWFRQAPGKERDFVATISSSGTNYANSVKGRFTISRDNAKNTVYLQMNSLRPEDTAVYYCNAKTVTRTGGVITGSRIEYDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 355 aa, Artificial sequence
  P56-10GS-Fc
EVQLVESGGGLVQPGGSLRLSCAASRSIYNVMAMGWYRQAPGKERELVAVVWSSGNTNYADSVKGRFTVSRDNAKNTVYLQMNSLRPEDTAVYYCYSKSLIGTSRYEIWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 363 aa, Artificial sequence
  P74-10GS-Fc
EVQLVESGGGLVQPGGSLRLSCAASGSTFSIKSIGWYRQAPGKQRELVADITRGAGINYADSVKGRFTISRDNTKNTMYLQMNSLRPEDTAVYYCNVKTIKRTGGIMTGSKIEYDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 354 aa, Artificial sequence
  P81-10GS-Fc
DVQLVESGGGLVQPGGSLRLSCAASGFSFSSFALSWYRQAPGKERELVARITTTGATNYADSVKGRFTISRDNAKNTVYLQMNSLRPEDTAVYYCNARARLWSVFDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 362 aa, Artificial sequence
  P89-10GS-Fc
DVQLVESGGGLVQPGGSLRLSCAASGRAFSSYNMGWFRQAPGKEREFVATISSSGTNYADSVKGRFTISRDNAKNTVYLQMNSLRPEDTAVYYCNAKTVTRTGGVITGSRIEYDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 355 aa, Artificial sequence
  P114-10GS-Fc
DVQLVESGGGLVQPGGSLRLSCAASRSIYNVLAMGWYRQAPGKERELVAVVWSSGNTNYADSVKGRFTISRDNAKNTVYLQMNSLRPEDTAVYYCYSKSLIGTSRYEIWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 355 aa, Artificial sequence
  P115-10GS-Fc
DVQLVESGGGLVQPGGSLRLSCAASRSIYNVRAMGWYRQAPGKERELVAVVWSSGNTNYADSVKGRFTISRDNAKNTVYLQMNSLRPEDTAVYYCYSKSLIGTSRYEIWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 68 - protein, 363 aa, Artificial sequence
  P125-10GS-Fc
DVQLVESGGGLVQPGGSLRLSCAASGSTFSIKSIGWYRQAPGKQRELVADITRGAGINYADSVKGRFTISRDNAKNTVYLQMNSLRPEDTAVYYCNVKTIKRTGGILTGSKIEYDYWGQGTLVTVSSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 364 aa, Artificial sequence
  P62-20GS-Fc
EVQLVESGGGLVQPGGSLRLSCAASGFSFSSFALSWYRQAPGKERELVARITTTGATNYADSVKGRFTISRDNAKNMGYLQMNSLRPEDTAVYYCNARARLWSVFDYSGQGTLVTVSSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 70 - protein, 372 aa, Artificial sequence
  P65-20GS-Fc
EVQLVESGGGLVQPGGSLRLSCAASGRAFSSYNMGWFRQAPGKERDFVATISSSGTNYANSVKGRFTISRDNAKNTVYLQMNSLRPEDTAVYYCNAKTVTRTGGVITGSRIEYDYWGQGTLVTVSSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 365 aa, Artificial sequence
  P56-20GS-Fc
EVQLVESGGGLVQPGGSLRLSCAASRSIYNVMAMGWYRQAPGKERELVAVVWSSGNTNYADSVKGRFTVSRDNAKNTVYLQMNSLRPEDTAVYYCYSKSLIGTSRYEIWGQGTLVTVSSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 72 - protein, 373 aa, Artificial sequence
  P74-20GS-Fc
EVQLVESGGGLVQPGGSLRLSCAASGSTFSIKSIGWYRQAPGKQRELVADITRGAGINYADSVKGRFTISRDNTKNTMYLQMNSLRPEDTAVYYCNVKTIKRTGGIMTGSKIEYDYWGQGTLVTVSSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 364 aa, Artificial sequence
  P81-20GS-Fc
DVQLVESGGGLVQPGGSLRLSCAASGFSFSSFALSWYRQAPGKERELVARITTTGATNYADSVKGRFTISRDNAKNTVYLQMNSLRPEDTAVYYCNARARLWSVFDYWGQGTLVTVSSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 372 aa, Artificial sequence
  P89-20GS-Fc
DVQLVESGGGLVQPGGSLRLSCAASGRAFSSYNMGWFRQAPGKEREFVATISSSGTNYADSVKGRFTISRDNAKNTVYLQMNSLRPEDTAVYYCNAKTVTRTGGVITGSRIEYDYWGQGTLVTVSSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 365 aa, Artificial sequence
  P114-20GS-Fc
DVQLVESGGGLVQPGGSLRLSCAASRSIYNVLAMGWYRQAPGKERELVAVVWSSGNTNYADSVKGRFTISRDNAKNTVYLQMNSLRPEDTAVYYCYSKSLIGTSRYEIWGQGTLVTVSSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 365 aa, Artificial sequence
  P115-20GS-Fc
DVQLVESGGGLVQPGGSLRLSCAASRSIYNVRAMGWYRQAPGKERELVAVVWSSGNTNYADSVKGRFTISRDNAKNTVYLQMNSLRPEDTAVYYCYSKSLIGTSRYEIWGQGTLVTVSSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 373 aa, Artificial sequence
  P125-20GS-Fc
DVQLVESGGGLVQPGGSLRLSCAASGSTFSIKSIGWYRQAPGKQRELVADITRGAGINYADSVKGRFTISRDNAKNTVYLQMNSLRPEDTAVYYCNVKTIKRTGGILTGSKIEYDYWGQGTLVTVSSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 226 aa, Artificial sequence
  IgG1 short hinge
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 79 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 80 - protein, 366 aa, Artificial sequence
  hCCR8-delta18-3XHA
MYPYDVPDYAAYPYDVPDYAGYPYDVPDYAIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 81 - protein, 5 aa, Artificial sequence
  CDR1-Kabat-P62/P81
SFALS

SEQ ID NO: 82 - protein, 5 aa, Artificial sequence
  CDR1-Kabat-P65/P89
SYNMG

SEQ ID NO: 83 - protein, 5 aa, Artificial sequence
  CDR1-Kabat-P56
VMAMG

SEQ ID NO: 84 - protein, 5 aa, Artificial sequence
  CDR1-Kabat-P74/P125
IKSIG

SEQ ID NO: 85 - protein, 5 aa, Artificial sequence
  CDR1-Kabat-P114
VLAMG

SEQ ID NO: 86 - protein, 5 aa, Artificial sequence
  CDR1-Kabat-P115
VRAMG

SEQ ID NO: 87 - protein, 10 aa, Artificial sequence
  CDR3-Kabat-P62
RARLSSVFDY

SEQ ID NO: 88 - protein, 10 aa, Artificial sequence
  CDR3-Kabat-P81
RARLWSVFDY

SEQ ID NO: 89 - protein, 19 aa, Artificial sequence
  CDR3-Kabat-P65//P89
KTVTRTGGVITGSRIEYDY

SEQ ID NO: 90 - protein, 11 aa, Artificial sequence
  CDR3-Kabat-P56/P114/P115
KSLIGTSRYEI

SEQ ID NO: 91 - protein, 19 aa, Artificial sequence
  CDR3-Kabat-P74
KTIKRTGGIMTGSKIEYDY

SEQ ID NO: 92 - protein, 19 aa, Artificial sequence
  CDR3-Kabat-P125
KTIKRTGGILTGSKIEYDY

SEQ ID NO: 93 - protein, 98 aa, Homo sapiens
EVQLVESGGGLVQPGGSLRLSCAASGFTFSSYAMHWVRQAPGKGLEWVSVISSDGSSTYYADSVKGRFTISRDNSKNTLYLQMNSLRAEDTAVYYCAR

SEQ ID NO: 94 - protein, 11 aa, Homo sapiens
WGQGTLVTVSS